(19) United States
(12) Patent Application Publication
(10) Pub. No.: US 2011/0111497 A1
(43) Pub. Date: May 12, 2011

(54) CELL SEPARATION APPARATUS, METHOD FOR ACTIVATING FAT-DERIVED CELLS, GRAFT MATERIAL PRODUCING PROCESS, AND GRAFT MATERIAL

(75) Inventors: Masato TAMAI, Tokyo (JP); Ayako KOHORI, Tokyo (JP); Takahiro OHNO, Tokyo (JP); Akihiro HORII, Tokyo (JP); Konghua LIN, Tokyo (JP)

(73) Assignee: OLYMPUS CORPORATION, Tokyo (JP)

(21) Appl. No.: 12/917,884

(22) Filed: Nov. 2, 2010

(30) Foreign Application Priority Data
Nov. 6, 2009 (JP) 2009-255053
Feb. 25, 2010 (JP) 2010-041117
Feb. 25, 2010 (JP) 2010-041118
Apr. 16, 2010 (JP) 2010-095127
Jul. 2, 2010 (JP) 2010-152209

(51) Int. Cl. C12N 5/071; C12M 1/00; C12M 1/36; C12M 1/38

(52) U.S. Cl. 435/366; 435/283.1; 435/286.5

(57) ABSTRACT

The therapeutic effect of a final product is improved while guaranteeing sterility. Provided is a cell separation apparatus comprising a decomposition treatment unit for producing a cell suspension by digesting living body tissue to release living body-derived cells from the living body tissue; a cell concentrating unit for generating a cell-concentrated solution by concentrating the cell suspension by centrifugation; and a efficiency-increasing mechanism for increasing the efficiency of the living body-derived cells contained in the cell-concentrated solution.

### BACKGROUND OF THE INVENTION

1. Field of the Invention

The present invention relates to a cell separation apparatus, a method for activating fat-derived cells, a graft material producing process, and a graft material.

This application is based on Japanese Patent Application Nos. 2009-255053, 2010-041117, 2010-041118, 2010-095127, and 2010-152209, the contents of which are incorporated herein by reference.

2. Description of Related Art

Cell separation apparatuses for separating fat-derived cells, including, for example, stem cells, from fat tissue collected from living bodies have been conventionally known (for example, see Japanese Unexamined Patent Application, Publication No. 2009-136168, PCT International Publication No. WO 2005/012480, and U.S. Patent Application Publication No. 2009/0304644). The separated fat-derived cells are used for therapy by, for example, being transplanted into the body of the donor of the fat tissue as a graft material.

Some fat-derived cells separated with the apparatus described in Japanese Unexamined Patent Application, Publication No. 2009-136168 or PCT International Publication No. WO 2005/012480 show decreased function in treatment or contain erythrocytes mixed therein. Treatment for removing erythrocytes or activating fat-derived cells may be additionally performed by taking out the fat-derived cells from the cell separation apparatus during or after the treatment process. However, in order to guarantee the sterility of the final product, it is desirable to perform the entire treatment process in a closed system, which does not need to be operated by a person.

### BRIEF SUMMARY OF THE INVENTION

It is an object of the present invention to provide a cell separation apparatus that can improve the therapeutic effect of a final product while guaranteeing its sterility, a method for activating fat-derived cells, a graft material producing process, and a graft material.

In order to achieve the above-mentioned object, the present invention provides the following solutions.

A first aspect of the present invention relates to a cell separation apparatus including a decomposition treatment unit for producing a cell suspension by digesting living body tissue to release living body-derived cells from the living body tissue; a cell concentrating unit for generating a cell-concentrated solution by concentrating the cell suspension by centrifugation; and a efficiency-increasing mechanism for increasing the efficiency of the living body-derived cells contained in the cell-concentrated solution.

A second aspect of the present invention relates to a method for activating fat-derived cells including heating fat-derived cells separated from human fat tissue at 38 to 42° C.

A third aspect of the present invention relates to a process of producing a graft material containing fat-derived cells separated from human fat tissue. The process includes the step of activating the fat-derived cells by heating the fat-derived cells at 38 to 42° C.

A fourth aspect of the present invention provides a graft material produced by the above-mentioned process of producing a graft material.

### BRIEF DESCRIPTION OF THE SEVERAL VIEWS OF THE DRAWINGS

FIG. 1 is an overall configuration diagram of a cell separation apparatus according to a first embodiment of the present invention.

FIG. 2 is a diagram illustrating a modification of the cell separation apparatus of FIG. 1.

FIG. 3 is a diagram illustrating another modification of the cell separation apparatus of FIG. 1 and showing a configuration having an erythrocyte-separating portion.

FIG. 4 is an overall configuration diagram of a cell separation apparatus according to a second embodiment of the present invention.

FIG. 5 is a graph showing the relationship between temperature and the number of activated fat-derived cells.

FIG. 6 is a graph showing the relationship between warming time and the number of activated fat-derived cells.

FIG. 7 shows a modification of the cell separation apparatus of FIG. 4 and is a diagram illustrating a configuration for activating fat-derived cells with ultrasound.

FIG. 8 shows another modification of the cell separation apparatus of FIG. 4 and is a diagram illustrating a configuration for activating fat-derived cells by rendering a cell suspension hypoxic.

FIG. 9 is an overall configuration diagram of a cell separation apparatus according to a third embodiment of the present invention.

FIG. 10 is a diagram illustrating a modification of a cell-stimulating container.

FIG. 11 is an overall configuration diagram of a cell separation apparatus according to a fourth embodiment of the present invention.

FIG. 12 is an overall configuration diagram of a cell separation apparatus according to a fifth embodiment of the present invention.

FIG. 13 is diagram illustrating the configuration of a centrifuge of the cell separation apparatus of FIG. 12.

FIG. 14 is a diagram illustrating the configuration of a centrifuge container used in the centrifuge of FIG. 13.

FIG. 15 is a diagram illustrating a receiving portion provided in the centrifuge of FIG. 13.

FIG. 16 is a diagram showing the configuration of a cell-counting portion and a method of counting the number of fat-derived cells with the cell-counting portion.

FIG. 17 is a diagram showing a method of measuring the three-dimensional shape of a cell clump with the cell-counting portion.

FIG. 18 is a diagram illustrating the configuration of a cell-stimulating portion, showing the arrangement of a heater and a temperature sensor.

FIG. 19 is a diagram illustrating another modification of the arrangement of the heater and the temperature sensor shown FIG. 18.

FIG. 20 is a diagram illustrating another modification of the arrangement of the heater and the temperature sensor shown FIG. 18.

FIG. 21 is a diagram illustrating a modification of the cell-stimulating portion.

FIG. 22 is a diagram illustrating a modification of the receiving portion.

FIG. 23 is a diagram illustrating another modification of the receiving portion.

FIG. 24 is a diagram illustrating a modification of the arrangement of the cell-stimulating portion.

FIG. 25 is a diagram illustrating a modification of the cell-counting portion.

FIG. 26 is a diagram illustrating another modification of the configuration of the cell-counting portion.

FIG. 27 is a diagram illustrating another modification of the configuration of the cell-counting portion.

FIG. 28 is a diagram illustrating another modification of the arrangement of the cell-counting portion.

FIG. 29 is a diagram illustrating another modification of the arrangement of the cell-counting portion.

FIG. 30 is an overall configuration diagram of a cell separation apparatus according to a sixth embodiment of the present invention.

FIG. 31 is a diagram illustrating the configuration of the cell-counting portion of the cell separation apparatus of FIG. 30.

FIG. 32 is a diagram illustrating the configuration of the cell-stimulating portion of the cell separation apparatus of FIG. 30.

FIG. 33 is a diagram illustrating a modification of the cell-counting portion of FIG. 31.

FIG. 34 is a diagram illustrating a modification of the cell separation apparatus of FIG. 30.

FIG. 35 is an overall configuration diagram of a cell separation apparatus according to a seventh embodiment of the present invention.

FIG. 36 is a diagram illustrating a modification of a cell-sorting channel of the cell separation apparatus of FIG. 35.

FIG. 37 is a diagram showing a method of sorting fat-derived cells with the cell-sorting channel of FIG. 36.

FIG. 38 is a diagram illustrating another modification of the cell-sorting channel of the cell separation apparatus of FIG. 35.

FIG. 39 is a flow chart of a graft material producing process according to the sixth embodiment of the present invention.

FIG. 40 is an overall configuration diagram illustrating a graft material producing apparatus for implementing the graft material producing process according to the sixth embodiment.

FIG. 41 is a graph showing the relationship between warming temperature and the number of wandering cells when fat-derived cells are heated.

FIG. 42 is a graph showing the relationship between warming treatment time and the number of wandering cells when fat-derived cells are heated at 40° C.

### DETAILED DESCRIPTION OF THE INVENTION

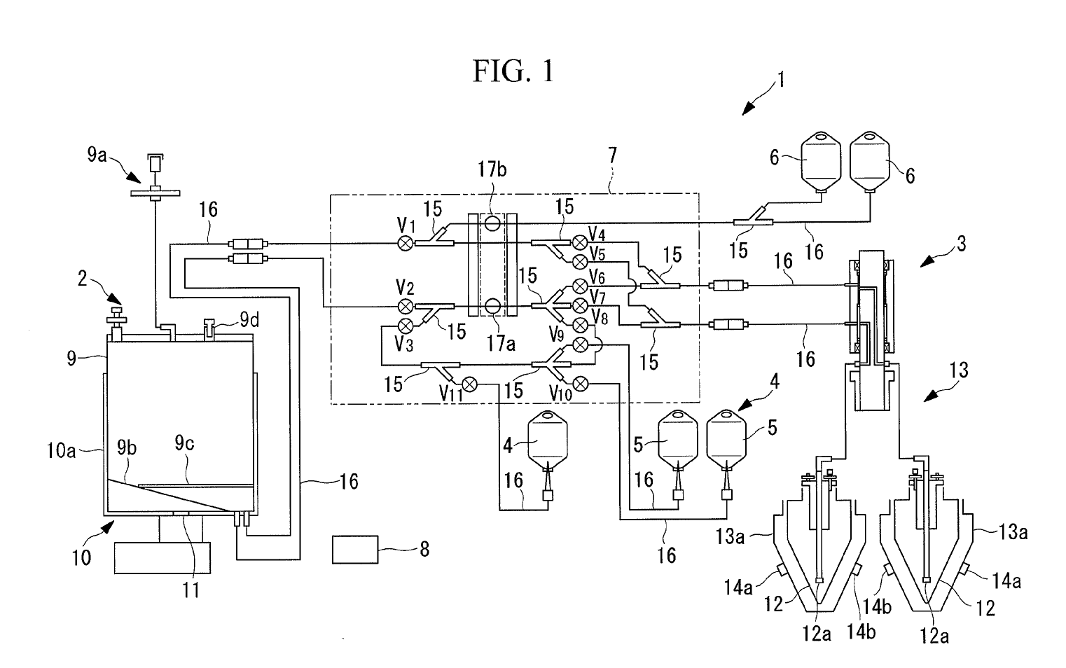

A first embodiment of the present invention will be described below with reference to FIGS. 1 to 3.

As shown in FIG. 1, a cell separation apparatus 1 according to the first embodiment includes a decomposition treatment unit 2 producing a cell suspension of fat-derived cells (living body-derived cells) by digesting fat tissue (living body tissue); a cell concentrating unit 3 washing and concentrating the cell suspension by centrifugation; a medium bag (medium-supplying mechanism) 4 containing a density-gradient solution; a washing solution bag 5 containing a washing solution; a waste solution bag 6 into which waste solution generated in the decomposition treatment unit 2 and the cell concentrating unit 3 is discharged; a conveyance path system 7 conveying solutions among the decomposition treatment unit 2, the cell concentrating unit 3, and the bags 4, 5, and 6; and a computer (medium supply amount calculator, controller, recovery mechanism, discharge-amount adjuster) 8 controlling the operation of the decomposition treatment unit 2, the cell concentrating unit 3, and the conveyance path system 7.

The decomposition treatment unit 2 includes a collecting container (receiving container) 9 collecting fat tissue, a shaker (stirring mechanism) 10 shaking the collecting container 9, and a load cell (content-measuring portion, scale) 11 provided in the shaker 10.

The collecting container 9 collects fat tissue suctioned from a living body with a cannula (not shown) connected to an inlet port 9a, which is provided at the upper portion of the collecting container 9. A bottom face 9b of the collecting container 9 inclines to one side and has a discharge port at the lowest position thereof. The discharge port is covered with a fat tissue-capturing filter 9c capturing fat tissue and allowing fat-derived cells to pass through. The collecting container 9 is supplied with a digestive enzyme solution from an enzyme supply port 9d provided at the upper portion of the collecting container 9 using, for example, a syringe, which is operated by an operator.

The shaker 10 has a base 10a approximately horizontally supporting the collecting container 9 and stirs the contents in the collecting container 9 by translational rotation of the base 10a in the horizontal plane.

The load cell 11 is provided on the base 10a at the position at which the base 10a supports approximately the center of the bottom of the collecting container 9 and measures the weight of the collecting container 9 set to the shaker 10.

Fat tissue collected in the collecting container 9 is digested by being stirred together with the digestive enzyme solution with the shaker 10. By doing so, the fat-derived cells that are bound to the fat tissue are released into the digestive enzyme solution to produce a cell suspension. By leaving the collecting container 9 to stand after stirring, the produced cell suspension and the residue of the fat tissue are separated into layers and the produced cell suspension forms a lower layer. By conducting discharge in this state, the cell suspension is selectively discharged from the collecting container 9 through the discharge port.

The cell concentrating unit 3 includes a pair of centrifuge containers 12, a centrifuge 13 rotating the centrifuge containers 12 so as to turn the bottom portions outward in the radial direction, and absorbance sensors (recovery mechanism) 14 measuring absorbance at different positions in the depth direction in each centrifuge container 12.

The centrifuge containers 12 receive the cell suspension discharged from the collecting container 9 through the conveyance path system 7. The centrifuge containers 12 are each provided with a pipe (discharge path) 12a extending from the upper portion to near the bottom portion of the centrifuge container 12, and the solution in the centrifuge container 12 is sequentially discharged from the lower layer side by suctioning the inside of the pipe 12a by means of the conveyance path system 7.

Each centrifuge container 12 may further have another pipe (not shown), in addition to the pipe 12a. The additional pipe extends to a position farther away from the bottom portion compared with the position of the pipe 12a and is provided with, for example, a two-way valve (not shown) so that one of the pipes is selected. By doing so, after precipitation of fat-derived cells by centrifugation of the cell suspension, the supernatant can be discharged through the additional pipe, while keeping the fat-derived cells at the bottom portion of the centrifuge container 12. The agglutinated fat-derived cells on the bottom portion are stirred in a washing solution by the force of the inflowing washing solution when the washing solution is sequentially supplied through the pipe 12a.

The absorbance sensors 14 each include a light-emitting portion 14a and a light-receiving portion 14b disposed so as to have a basket 13a supporting the centrifuge container 12 therebetween in the diameter direction of the basket 13a (hereinafter, also referred to as absorbance sensor 14a, 14b). The absorbance sensors 14 each measure the absorbance inside the centrifuge container 12 by emitting detection light from the light-emitting portion 14a toward the light-receiving portion 14b and detecting the intensity of the detection light passing through the centrifuge container 12 and being received by the light-receiving portion 14b.

During this process, the light-emitting portion 14a and the light-receiving portion 14b measure the absorbance at a plurality of positions in the depth direction of the centrifuge container 12 by emitting and receiving laser light, while moving in the vertical direction, for example, along guide rails (not shown). In order to allow the detection light emitted from the light-emitting portion 14a to reach the light-receiving portion 14b, the centrifuge container 12 is made of, for example, transparent plastic, and the basket 13a is provided with window (not shown) that is made of, for example, transparent glass.

The medium bag 4 contains a density-gradient solution having high biocompatibility. The density-gradient solution has a specific gravity larger than that of fat-derived cells and smaller than that of erythrocytes. The specific gravity of the density-gradient solution is preferably from 1.050 to 1.100 at 20° C. The density-gradient solution may be, for example, a commercially available product such as Ficoll, Percoll, Hypaque, or Pelcoll (products of GE Healthcare Japan) or may be a solution of a saccharide, such as sucrose, or cesium chloride prepared by adjusting the specific gravity to an appropriate level.

The density-gradient solution is supplied into the centrifuge containers 12 containing the cell suspension, and then centrifugation is performed by the centrifuge 13. As a result, among the components contained in the cell suspension, components, including erythrocytes, having specific gravity larger than that of the density-gradient solution are fractionated lower than the density-gradient solution and form a layer (hereinafter referred to as erythrocyte layer). On the other hand, other components, including fat-derived cells, in the cell suspension are fractionated higher than the density-gradient solution and form a layer (hereinafter referred to as fat-derived cell layer). Subsequently, the erythrocyte layer is first discharged through the pipes 12a, and other layers are sequentially discharged. The conditions for the centrifugation depend on, for example, the specific gravity of the density-gradient solution, but are preferably, for example, a centrifugal force of 400×g for about 30 minutes.

The washing solution bag 5 contains, for example, physiological saline or lactated Ringer buffer as a washing solution.

The conveyance path system 7 includes tubes 16 connecting each of the containers 9 and 12 and each of the bags 4, 5, and 6 through joints 15; a supply pump 17a and a drainage pump 17b disposed at intermediate points in the tubes 16 and allowing noncontact pumping of the solution in the tubes 16; and valves V1 to V11 switching the paths for supplying solution with the pumps 17a and 17b.

The computer 8 includes a storage unit and a central processing unit (CPU), and a program produced according to the fat tissue treatment process is stored in the storage unit. The computer 8 is connected to the shaker 10, the load cell 11, the centrifuge 13, the absorbance sensors 14, the pumps 17a and 17b, and the valves V1 to V11 via wiring (not shown) and operates them according to the program.

During this process, the computer 8 calculates the weight of the cell suspension supplied in the centrifuge containers 12 from the difference in weights measured by the load cell 11 before and after sending the cell suspension from the collecting container 9 to the centrifuge containers 12. Then, based on the calculated weight of the cell suspension, the computer 8 sets the length of time for sending the density-gradient solution from the medium bag 4 to the centrifuge containers 12, that is, the supply amount of the density-gradient solution.

In addition, the computer 8 stores absorbance values of an erythrocyte layer, a density-gradient solution layer, and a fat-derived cell layer, measured by the absorbance sensors 14 in advance. The computer 8 controls the centrifuge 13 to centrifuge the cell suspension and the density-gradient solution supplied in the centrifuge containers 12 and controls the absorbance sensors 14 to measure the absorbance inside centrifuge containers 12 after completion of centrifugation. The computer 8 detects the height position of the interface between the density-gradient solution and the fat-derived cell layer by comparing the measured absorbance and the stored absorbance values. Then, the computer 8 calculates the solution amount of the erythrocyte layer and the density-gradient solution layer based on the detected height position of the interface and sets the length of time for the subsequent discharge from the centrifuge containers 12 so that the discharged amount is equal to the calculated solution amount.

The operation and effects of the thus-configured cell separation apparatus 1 will be described below.

The cell separation apparatus 1 according to this embodiment supplies the washing solution into the collecting container 9 from the washing solution bag 5 when fat tissue is collected in the collecting container 9, washes the fat tissue by stirring it together with the washing solution, and discharges the washing solution used for washing to the waste solution bag 6.

Subsequently, the cell separation apparatus 1 produces a cell suspension by stirring the fat tissue and the digestive enzyme solution supplied in the collecting container 9 by an operator. The cell separation apparatus 1 conveys the cell suspension to the centrifuge containers 12 and supplies a density-gradient solution in an amount according to the weight of the cell suspension to the centrifuge containers 12. Then, the cell separation apparatus 1 fractionates erythrocytes contained in the cell suspension by centrifugation and then discharges the erythrocyte layer and the density-gradient solution layer from the centrifuge containers 12, while leaving the fat-derived cell layer.

Subsequently, the cell separation apparatus 1 supplies a washing solution to the centrifuge containers 12, precipitates the fat-derived cells by centrifugation, and discharges the supernatant in each centrifuge container 12 from another pipe. The process from the supply of the washing solution to the discharge of the supernatant is repeated few times to remove the digestive enzyme solution contained in the cell suspension and to wash the fat-derived cells. After completion of the washing, the cell clump precipitated by the centrifugation is collected using, for example, a syringe to obtain a concentrate of fat-derived cells, which has been sufficiently washed and from which erythrocytes have been sufficiently removed.

Thus, according to this embodiment, an advantage is afforded in that a concentrate of highly purified fat-derived cells can be obtained by sufficiently removing erythrocytes, which are difficult to remove only by stirring and centrifugation, by fractionating fat-derived cells and erythrocytes using a density-gradient solution. In addition, an appropriate supply amount of the density-gradient solution is calculated from the weight of the cell suspension measured by the load cell 11, and an appropriate discharge amount from the centrifuge containers 12 is calculated using the absorbance sensors 14. By doing so, all treatment can be performed in a noncontact manner with a sample and in a closed system, which provides an advantage in that the sterility of the final product is guaranteed.

In this embodiment, the weight of the cell suspension is calculated based on the weight of the collecting container 9. Instead of this, the weight of the cell suspension may be measured by disposing a weight-measuring container and a scale measuring the weight of the weight-measuring container between the collecting container 9 and the centrifuge containers 12, temporarily storing the cell suspension in the weight-measuring container on its way from the collecting container 9 to the centrifuge containers 12, and measuring the weight. By doing so, an appropriate supply amount of the density-gradient solution can be calculated.

In this embodiment, the supply amount of the density-gradient solution is calculated based on the weight of the cell suspension, but, instead of this, it may be calculated based on the volume of the cell suspension. As means for detecting the volume of a cell suspension, an interface sensor (content-measuring portion) detecting the height position of the interface between the cell suspension and the layer of residue of the fat tissue separated in the collecting container 9 or the height position of the interface of the cell suspension in the centrifuge containers 12 is used. For example, an optical, ultrasonic, or magnetic interface sensor is used.

When an optical interface sensor is used, as in the case of the absorbance sensors 14, the height position of the interface of a cell suspension is detected by measuring the absorbance or transmittance at different positions in the depth direction using a light-emitting portion and a light-receiving portion disposed on the outer side of the container 9 or 12. When an ultrasonic or magnetic interface sensor is used, the height position of the interface of a cell suspension is detected by inserting a probe emitting ultrasound or a magnetic field into the container 9 or 12 from above and detecting the ultrasound or magnetic field returning by reflection at the surface of the solution. By doing so, an appropriate supply amount of the density-gradient solution can be calculated.

In this embodiment, an absorbance sensor measuring the concentration of erythrocytes contained in a cell suspension may be provided, and the supply amount of the density-gradient solution may be calculated based on the weight of the cell suspension and the erythrocyte concentration measured by the absorbance sensor. In such a case, the erythrocyte concentration may be measured, before the supply of the density-gradient solution, using the absorbance sensors 14 provided on the baskets 13a of the centrifuge 13. Alternatively, the erythrocyte concentration may be measured while conveying the cell suspension from the collecting container 9 to the centrifuge containers 12 by separately providing an absorbance sensor at some points in the tubes 16. By doing so, even when the amount of erythrocytes suctioned together with fat tissue varies or the amount of erythrocytes remaining in a cell suspension varies due to a difference in efficiency of washing the fat tissue, erythrocytes can be more reliable removed by supplying a more appropriate amount of the density-gradient solution.

In this embodiment, the density-gradient solution is added to the cell suspension in the cell concentrating unit 3. Instead of this, the density-gradient solution may be added to the cell suspension in the decomposition treatment unit 2. In such a case, the density-gradient solution may be added in the collecting container 9 subsequently, after digestion of fat tissue, or the cell suspension washed by centrifugation may be conveyed to a collecting container 9 that has been newly changed and the density-gradient solution may be added thereto. The absorbance sensor 14 is disposed on the outer side of the collecting container 9.

After the addition of the density-gradient solution to the cell suspension in the collecting container 9, the collecting container 9 is left to stand for a predetermined time to fractionate the mixture into a fat-derived cell layer, a density-gradient solution layer, and an erythrocyte layer. Subsequently, the interface between the fat-derived cell layer and the density-gradient solution layer is detected with the absorbance sensor 14, and the erythrocyte layer and the density-gradient solution layer are discharged to obtain a cell suspension from which erythrocytes have been removed.

In this embodiment, a hemagglutinating agent may be used instead of the density-gradient solution. In such a case, the medium bag 4 contains the hemagglutinating agent.

The hemagglutinating agent is preferably a solution of a material that agglutinates erythrocytes in a chain form, such as hydroxyethylated starch, fibrinogen, α-globulin, chondroitin sulfate, or human serum albumin, while suppressing the influence on the fat-derived cells. By mixing the hemagglutinating agent with the cell suspension, the erythrocytes in the cell suspension agglutinate and precipitate in the cell suspension. Thus, an erythrocyte layer consisting of the agglutinated erythrocytes and a layer consisting of other components of the cell suspension (hereinafter, referred to as fat-derived cell layer) are formed.

In such a case, the computer 8 sets the supply amount of the hemagglutination agent based on the weight of the cell suspension measured by the load cell 11 and detects the height position of the interface between the fat-derived cell layer and the erythrocytes layer from the absorbance value measured by the absorbance sensor 14.

The thus-configured cell separation apparatus 1 produces a cell suspension in the collecting container 9, then sends the hemagglutinating agent from the medium bag 4 to the collecting container 9, and stirs the inside of the collecting container 9 for a predetermined time to allow erythrocytes to agglutinate by a reaction between the erythrocytes and the hemagglutinating agent.

Then, the cell separation apparatus 1 conveys the produced cell suspension to the centrifuge containers 12 and precipitates the agglutinated erythrocytes contained in the cell suspension by centrifugation. The conditions for the centrifugation on this occasion are preferably a centrifugal force of from 190 to 1190×g for 3 to 60 minutes. Subsequently, the cell separation apparatus 1 discharges the erythrocyte layer from the centrifuge containers 12, while retaining the fat-derived cell layer.

Thus, also by using the hemagglutinating agent, erythrocytes can be efficiently removed from the cell suspension to give highly purified fat-derived cells.

When the hemagglutinating agent is used, the hemagglutinating agent may be supplied after completion of washing treatment in the cell concentrating unit 3. In such a case, the cell suspension washed in the cell concentrating unit 3 may be conveyed to a collecting container 9 that has been newly changed, and the hemagglutinating agent may be supplied to the collecting container 9.

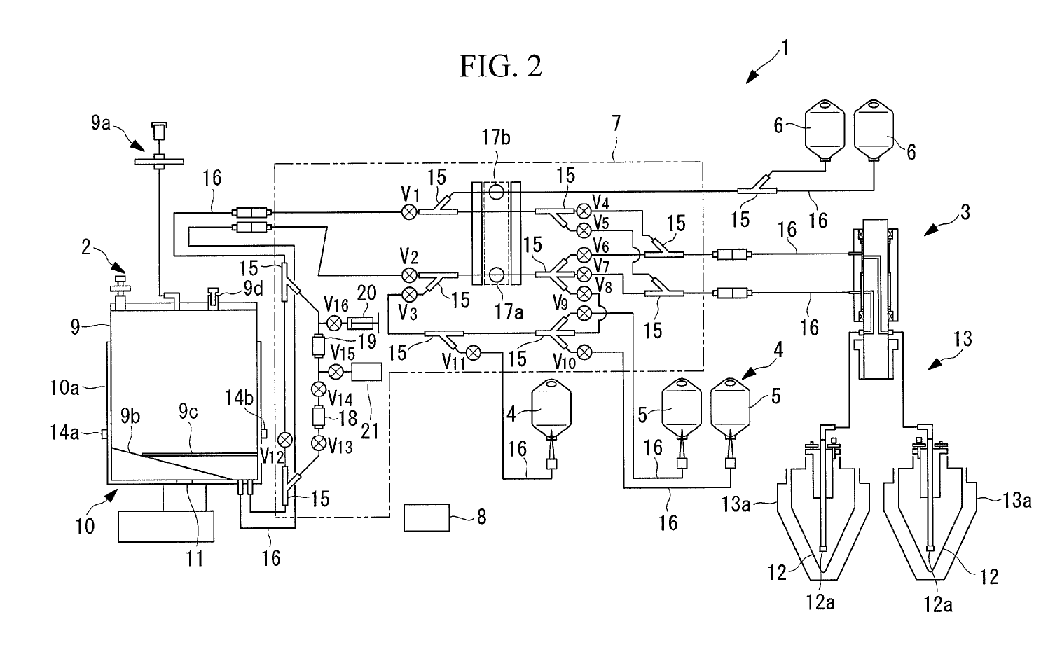

In the case where the erythrocytes are agglutinated in the collecting container 9 after completion of washing in the cell concentrating unit 3, as shown in FIG. 2, the cell separation apparatus 1 includes an erythrocyte-capturing filter 18 capturing agglutinated erythrocytes and allowing fat-derived cells to pass through, a cell-capturing filter 19 disposed at the subsequent stage of the erythrocyte-capturing filter 18 and capturing the fat-derived cells, a syringe 20 connected to the subsequent stage of the cell-capturing filter 19, and a recovery container 21 recovering a final product. The absorbance sensor 14 is disposed on the outer side of the collecting container 9.

The erythrocyte-capturing filter 18 and the cell-capturing filter 19 are disposed at an intermediate point in the discharge path from the collecting container 9 to the waste solution bag 6. The recovery container 21 is connected to the tube 16 between the filters 18 and 19 via a valve V15. The syringe 20 is connected to the tube 16 via a valve V16.

The pore size of the erythrocyte-capturing filter 18 is preferably from 30 to 500 μm. The erythrocyte-capturing filter 18 and the cell-capturing filter 19 are preferably subjected to hydrophilic treatment, such as modifying the surface with a material having a hydrophilic residue, in order to prevent adhesion of fat-derived cells.

The thus-configured cell separation apparatus 1 conveys the washed cell suspension from the centrifuge containers 12 to the collecting container 9, then supplies the hemagglutinating agent to the collecting container 9 from the medium bag 4, and stirs the cell suspension and the hemagglutinating agent with the shaker 10. The stirring time may be set by the computer 8 according to the weight of the cell suspension. Subsequently, only valves V1, V13, and V14 are opened to discharge the cell suspension from the collecting container 9 to the waste solution bag 6, and thereby the fat-derived cells from which erythrocytes are removed by the erythrocyte-capturing filter 18 are captured by the cell-capturing filter 19.

After completion of discharge of the cell suspension from the collecting container 9, the valves V1, V13, and V14 are closed, and the valves V15 and V16 are opened to forcibly apply a solution, such as washing solution, toward the cell-capturing filter 19 using the syringe 20. By doing so, the fat-derived cells captured by the cell-capturing filter 19 are released and flow into the recovery container 21 to obtain the fat-derived cells, from which erythrocytes are sufficiently removed, in the recovery container 21.

By doing so, erythrocytes can be efficiently and selectively removed with a simpler configuration and a simpler control method. Since the size of human erythrocyte is about 8 μm, in order to remove individual erythrocytes with a filter, the filter must have a pore size smaller than 8 μm and therefore has a disadvantage of becoming easily clogged. However, by agglutinating erythrocytes, the erythrocytes can be efficiently removed using the erythrocyte-capturing filter 18 having a considerably large pore size, which can eliminate the problems of clogging, etc.

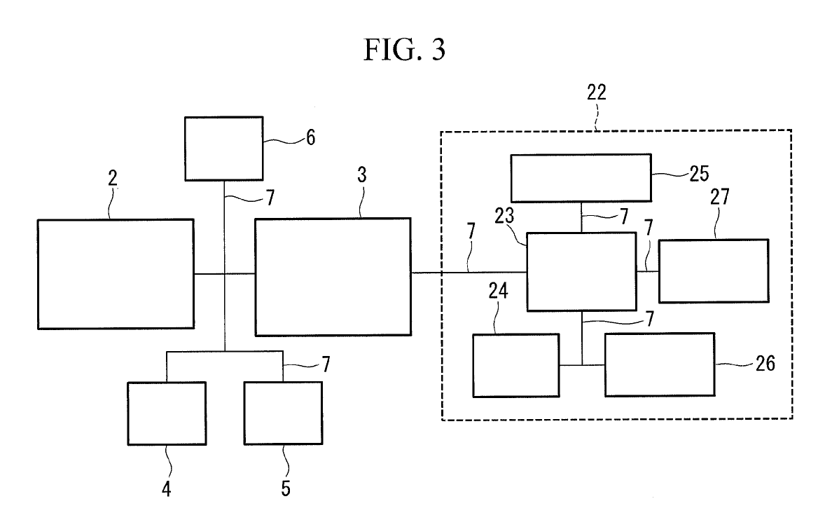

In the embodiment described above, a case where the cell suspension is reacted with the density-gradient solution or the hemagglutinating agent utilizing the configuration of the decomposition treatment unit 2 or the cell concentrating unit 3 has been described as an example; however, as shown in FIG. 3, an erythrocyte-removing portion 22 may be provided at the subsequent stage of the cell concentrating unit 3.

The erythrocyte-removing portion 22 includes a receiving container 23 receiving a cell suspension; a content-measuring portion 24 measuring the contents of the receiving container 23; a medium-supplying mechanism 25 supplying a density-gradient solution or a hemagglutinating agent into the receiving container 23; a medium reaction mechanism 26, such as a centrifuge or a stirrer, accelerating fractionating of erythrocytes and fat-derived cells in the receiving container 23; and a recovery mechanism 27 recovering the fraction of fat-derived cells from the receiving container 23. The amount of the density-gradient solution or the hemagglutinating agent supplied by the medium-supplying mechanism 25 is controlled by the computer 8 as in the embodiment described above. By doing so, effects similar to those in the above-described embodiment can be obtained.

In the above-described embodiment, a case where fat-derived cells are separated from fat tissue has been exemplified, but the cell separation apparatus 1 can be used for separating living body-derived cells from other living body samples containing erythrocytes, such as bone marrow fluid containing a large number of stem cells.

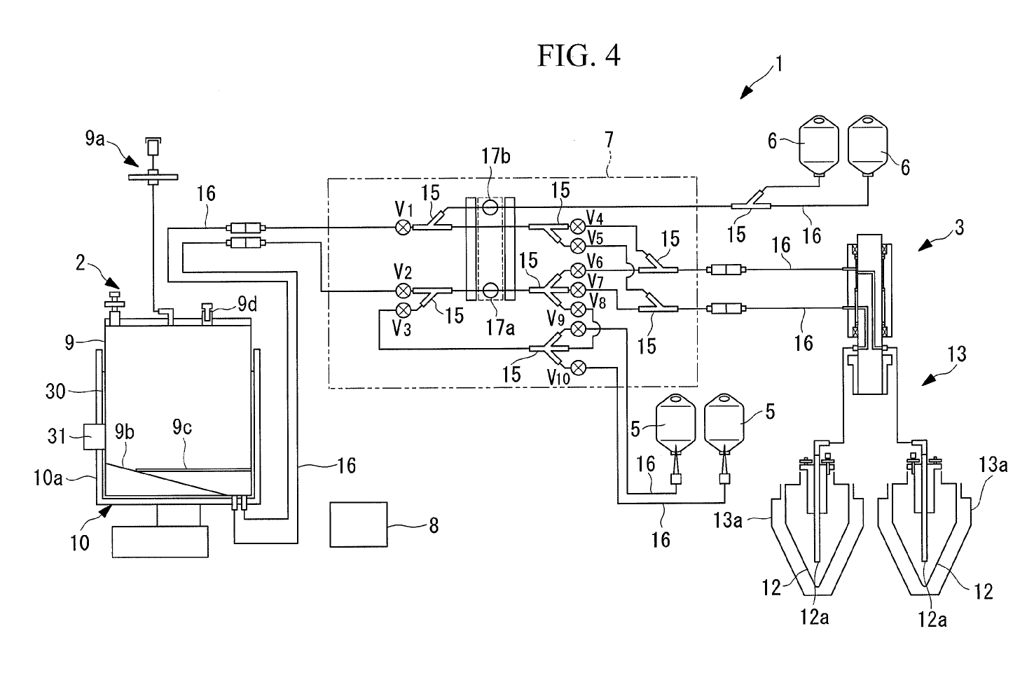

A cell separation apparatus 1 according to a second embodiment of the present invention will be described below with reference to FIGS. 4 to 8.

The same configurations as those in the above-described first embodiment are designated with the same reference numerals, and descriptions thereof are omitted. As shown in FIG. 4, the cell separation apparatus 1 according to this embodiment differs from that of the first embodiment mainly in the configuration of the decomposition treatment unit 2.

The decomposition treatment unit 2 includes a heater (cell-stimulating portion) 30 warming the inside of the collecting container 9 and a temperature sensor (state-quantity measuring portion) 31 measuring the temperature of the inside of the collecting container 9.

The heater 30, for example, has a sheet-like shape and is disposed on the inner face of a base 10a.

The temperature sensor 31 used is an infrared or thermocouple type. The temperature sensor 31 is disposed on a window provided so as to pass through the base 10a. When the infrared type is used, the temperature sensor 31 is arranged so as to have a gap between the temperature sensor 31 and the side wall of the collecting container 9. When the thermocouple type is used, the temperature sensor 31 is arranged so as to be in contact with the side wall of the collecting container 9.

The computer 8 is connected to the heater 30 and the temperature sensor 31 via wiring (not shown).

Here, when fat tissue is digested in the collecting container 9, the computer 8 controls the shaker 10 to stir the inside of the collecting container 9 for a predetermined time, while controlling the temperature of the heater 30 so that the temperature measured by the temperature sensor 31 is maintained at 37° C. Subsequently, the computer 8 controls the heater 30 to increase the temperature and controls the shaker 10 to stir the inside of the collecting container 9 for a predetermined time, preferably, for about 30 minutes, while controlling the temperature of the heater 30 so that the temperature measured by the temperature sensor 31 is maintained at 38 to 42° C., more preferably, at 40° C. During this process, an increase in the temperature measured by the temperature sensor 31, namely, a cell suspension temperature of 42° C. or higher, may deteriorate the function of the fat-derived cells due to, for example, agglutination of protein in the fat-derived cells and is therefore undesirable.

The operation and effects of the thus-configured cell separation apparatus 1 will be described below.

The cell separation apparatus 1 according to this embodiment produces a cell suspension by stirring fat tissue and a digestive enzyme solution in the collecting container 9, and then increases the temperature of the cell suspension from 37° C., and continues to stir the cell suspension while maintaining the temperature at 38 to 42° C. Then, after letting the collecting container 9 stand, the cell separation apparatus 1 conveys the cell suspension to the centrifuge containers 12. Subsequently, the cell separation apparatus 1 precipitates fat-derived cells by centrifuging the cell suspension, discharges the supernatant through the pipes 12a, and, according to need, washes the fat-derived cells. By doing so, a concentrate of the fat-derived cells is obtained.

In this case, according to this embodiment, the fat-derived cells in the cell suspension, whose function has been suppressed, are stimulated by further increasing the temperature of the cell suspension from 37° C., after the digestion of the fat tissue. By doing so, the fat-derived cells are activated to a state of increased activity, which provides an advantage in that a sufficient therapeutic effect can be promptly obtained even if the recovered fat-derived cells are directly used for therapy. Furthermore, by stimulating the fat-derived cells in a noncontact manner using heat, the fat-derived cells can be activated by a simple configuration and operation even in a closed system, which provides an advantage in that the sterility of the fat-derived cells can be guaranteed.

FIG. 5 shows the measurement results of activation status of fat-derived cells when their temperature is changed.

First, fat-derived cells separated from human fat tissue were kept at a predetermined temperature for 30 minutes as heat treatment. Subsequently, using a BD BioCoat Matrigel invasion chamber, manufactured by Becton Dickinson Japan, the number of fat-derived cells (hereinafter, referred to as wandering cells) that migrated into this chamber was counted. Specifically, 10% FBS culture medium containing 100 ng/mL of SDF-1 was placed in a well, and heat-treated fat-derived cells were put in a cell culture insert and were incubated for 24 hours. Then, the number of wandering cells that migrated from the cell culture insert to the well was counted.

The above-described experiment was performed using cell suspensions of fat-derived cells prepared so as to have approximately the same number of cells and subjecting them to heat treatment at 37, 38, 40, 42, or 43° C. In this experiment, the number of fat-derived cells that migrated by being attracted to SDF-1 and FBS due to the sufficiently high activity was counted, and the effect of activation by heat treatment was evaluated thereby. FIG. 5 shows the number of wandering cells counted at each temperature by converting it to a ratio relative to the number of wandering cells counted at 37° C. defined as 1. It was confirmed from FIG. 5 that the number of wandering cells increased with an increase in temperature from 37 to 40° C. and then decreased from 40° C., which is the maximum, and that the number at 43° C. was lower than that at 37° C. That is, it was confirmed that human fat-derived cells show the maximum activity at 40° C.

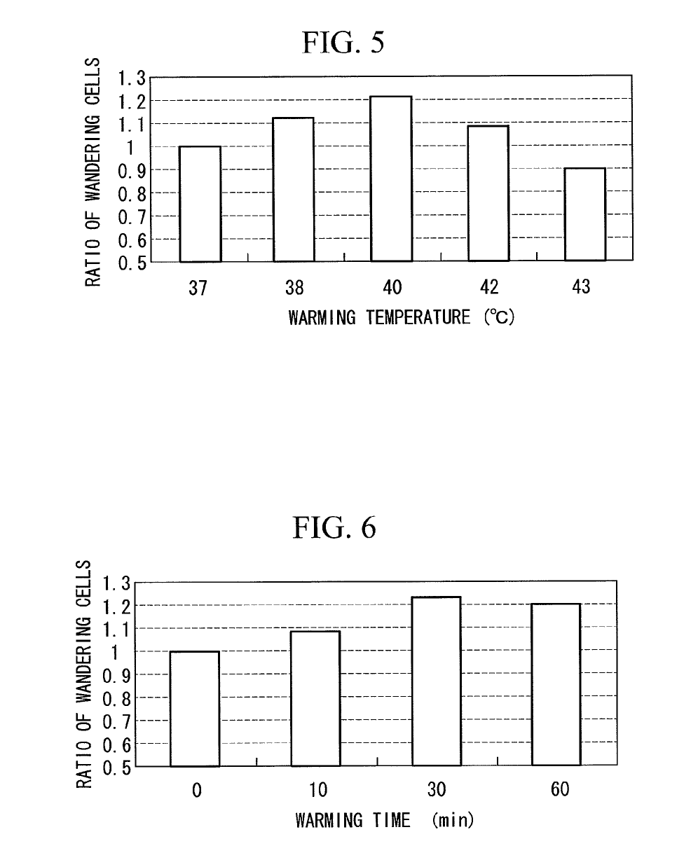

FIG. 6 shows the measurement results of the relationship between warming time and the number of wandering cells, when cell suspensions of fat-derived cells separated from human fat tissue were warmed at 40° C. The number of wandering cells was measured as in the experiment shown in FIG. 5. It was confirmed from FIG. 6 that the number of wandering cells increased with an increase in the length of time after being warmed at 40° C. and that sufficiently increased activity was obtained by being continuously warmed for 30 minutes.

In this embodiment, fat-derived cells are activated in the collecting container 9. Instead of this, fat-derived cells may be activated in the centrifuge containers (receiving containers) 12. In such a case, a heater and a temperature sensor (not shown) are separately provided on the basket 13a. By doing so, the time from the activation of fat-derived cells to their use for therapy can be shortened to enable the fat-derived cells to be used for therapy in a state where a higher activity is maintained.

In this embodiment, fat-derived cells are activated by warming them. Instead of this, fat-derived cells may be activated by being irradiated with ultrasound.

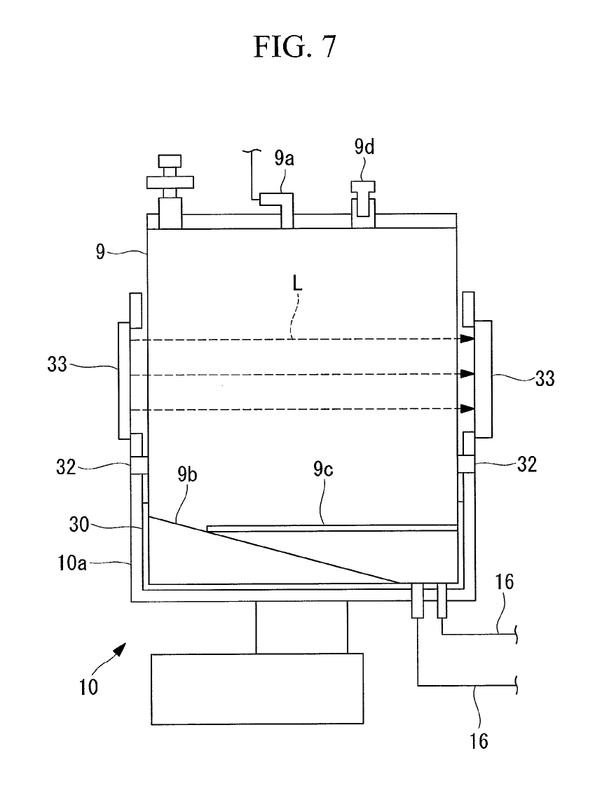

In such a case, as shown in FIG. 7, an ultrasonic transducer (cell-stimulating portion) 32 is disposed near the side face of the collecting container 9. Ultrasound is generated in the collecting container 9 by supplying high-frequency power to the ultrasonic transducer 32 from an ultrasound generator (not shown).

In this case, an interface sensor (state quantity-measuring portion) 33 detecting the interface of a cell suspension in the collecting container 9 is further provided. The interface sensor 33 emits laser light L in approximately horizontal direction at a plurality of positions in the depth direction of the collecting container 9 and receives the laser light L passed through the collecting container 9 at positions corresponding to the plurality of positions in the depth direction of the collecting container 9. By doing so, the interface sensor 33 detects the interface between the cell suspension and the residue of fat tissue from the difference in intensity of the laser light L received at each position.

After completion of the digestion of fat tissue in the collecting container 9 and after waiting for a predetermined time to allow the cell suspension and the residue of the fat tissue to separate into each layer in the collecting container 9, the computer 8 performs control so that the interface sensor 33 detects the depth position of the interface between the cell suspension and the residue of the fat tissue. The computer 8 sets the output level of ultrasound supplied to the ultrasonic transducer 32 from the ultrasound generator based on the detected depth position of the interface, and generates ultrasound in the collecting container 9.

By doing so, fat-derived cells can be activated in a noncontact state. Furthermore, by controlling the output level of ultrasound generated according to the amount of cell suspension, an appropriate stimulation intensity can be applied to the fat-derived cells.

As the interface sensor 33, for example, an ultrasonic or magnetic type may be used. In such a case, if the fat tissue residue is present on the upper layer of a cell suspension, the amount of cell suspension cannot be precisely measured.

Therefore, it is preferable to generate ultrasound by detecting the surface of a cell suspension in the centrifuge container 12 or in a separately provided receiving container (not shown).

Instead of the interface sensor 33, a weight sensor (not shown, state-quantity measuring portion) measuring the weight of the collecting container 9 may be provided, and the computer 8 may control the output level of ultrasound according to the weight of the contents in the collecting container 9 measured by the weight sensor.

In this embodiment, fat-derived cells may be activated by placing the fat-derived cells in a low-oxygen environment.

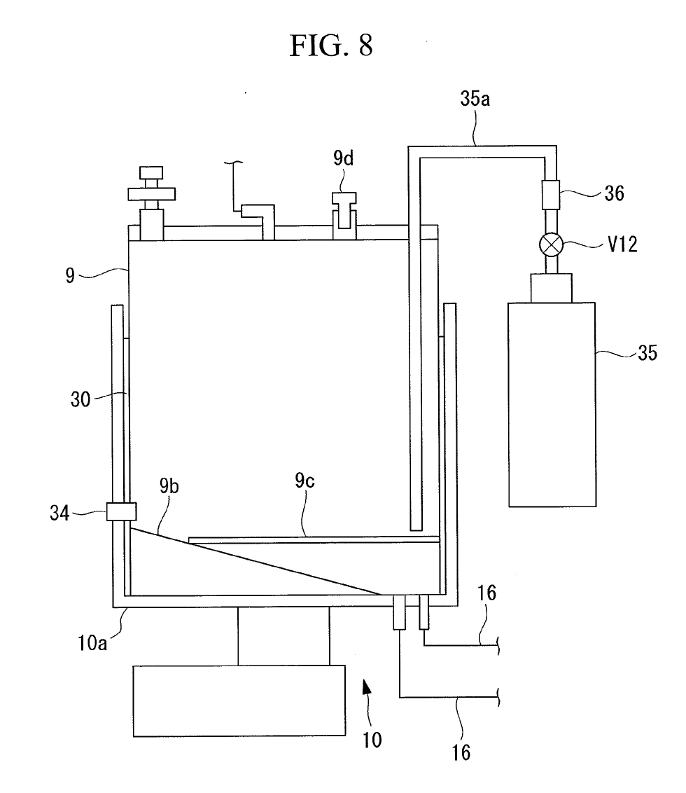

In this case, as shown in FIG. 8, a dissolved-oxygen meter (state-quantity measuring portion, oxygen concentration sensor) 34 is provided in the collecting container 9. In addition, a pipe 35a connected to a gas cylinder (cell-stimulating portion, low-oxygen-gas supplying portion) 35 is inserted into the collecting container 9 via a valve V12 so as to extend to near the bottom of the collecting container 9. The gas in the gas cylinder 35 is preferably a gas that effectively dilutes oxygen in a cell suspension, while suppressing the influence on the fat-derived cells, for example, an inert gas such as pure nitrogen. The computer 8 supplies the gas into the cell suspension by opening the valve V11 until the dissolved oxygen concentration measured by the dissolved oxygen meter 34 is decreased to a predetermined level. An air filter 36 for removing dust in the gas may be provided in the pipe 35a.

Alternatively, a washing solution having a reduced dissolved-oxygen content may be supplied to a cell suspension by disposing the washing solution bag 5, made of a gas permeable material, inside a sealed container (not shown) and making the inside of the container a low-oxygen atmosphere.

By doing so, the fat-derived cells can be activated by simple operation and configuration, while maintaining the sterility of the fat-derived cells.

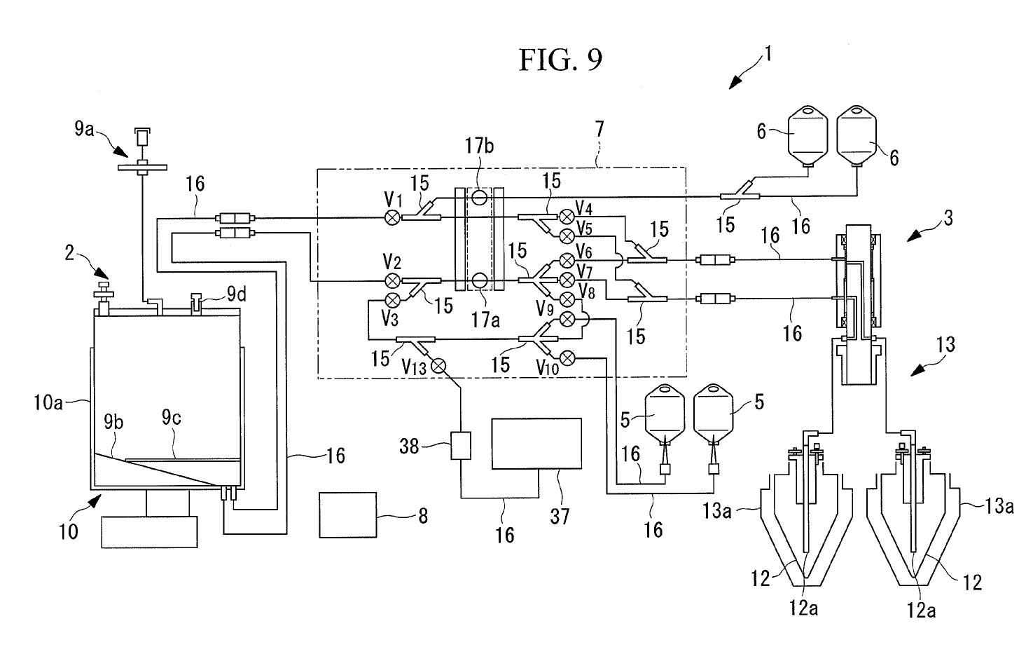

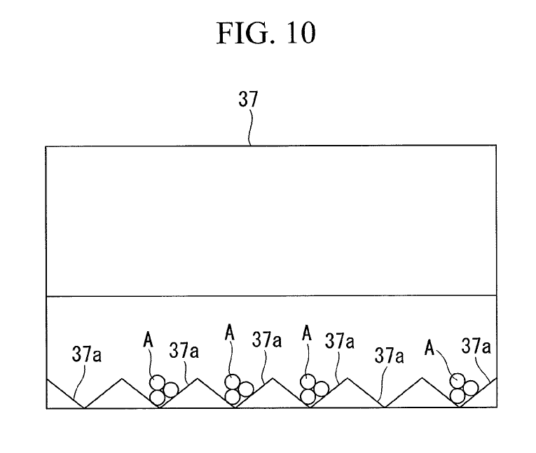

A third embodiment of the present invention will be described below with reference to FIGS. 9 and 10.

In this embodiment, the same configurations as those in the first and second embodiments are designated with the same reference numerals, descriptions thereof are omitted, and differences from the first and second embodiments will be mainly described.

As shown in FIG. 9, the cell separation apparatus 1 according to this embodiment includes a cell-stimulating container (receiving container, cell-stimulating portion) 37 and a turbidity sensor (state-quantity measuring portion, cell density meter) 38 disposed in the tube 16.

The cell-stimulating container 37 has an inner surface coated by a non-adhesive material so that fat-derived cells do not easily adhere to the surface. Examples of the non-adhesive material include silicone, phospholipid, polyhydroxyethyl methacrylate, and ethylene vinyl alcohol copolymers. By doing so, the fat-derived cells in a cell suspension received in the cell-stimulating container 37 cannot adhere to the inner wall and therefore adhere with one another to agglutinate.

The turbidity sensor 38 is arranged in the tube 16 conveying a cell suspension from the collecting container 9 to the cell-stimulating container 37 and measures the turbidity of the cell suspension passing through the tube 16. It is generally known that there is a proportional relationship between cell density and turbidity in a cell suspension.

The computer 8 stores the relationship between turbidity and density of the fat-derived cells by, for example, measuring the turbidity of a cell suspension whose fat-derived cell density has been determined in advance. By doing so, the computer 8 calculates the density of fat-derived cells contained in a cell suspension based on the turbidity measured by the turbidity sensor 38. Furthermore, the computer 8 calculates the amount of cell suspension to be supplied to the cell-stimulating container 37 based on the calculated density of fat-derived cells in advance for a predetermined number of fat-derived cells to be supplied to the cell-stimulating container 37, and the computer 8 sets the length of time for pumping the cell suspension with the pump 17.

The effects of the thus-configured cell separation apparatus 1 will be described below.

The cell separation apparatus 1 according to this embodiment sends a cell suspension produced in the collecting container 9 to the cell-stimulating container 37. Then, when the number of cells in the cell-stimulating container 37 reaches a predetermined value, the cell separation apparatus 1 stops sending the cell suspension. The cell separation apparatus 1 is left to stand for a predetermined time to let the fat-derived cells agglutinate with one another in the cell-stimulating container 37. Subsequently, the cell separation apparatus 1 conveys the cell suspension to the centrifuge containers 12 from the cell-stimulating container 37 to wash and concentrate the fat-derived cells.

In this case, according to this embodiment, the fat-derived cells are activated by coming into contact with one another at an appropriate number of cells in the cell-stimulating container 37. This provides an advantage in that fat-derived cells in a state of high activity can be obtained by simple configuration and operation, while maintaining a closed system.

In the above-described embodiment, the inner surface of the cell-stimulating container 37 is coated with a non-adhesive material. Instead of this, as shown in FIG. 10, grooves 37a may be formed in the bottom of the cell-stimulating container 37. By doing so, fat-derived cells A can be activated by being brought into contact with one another when they precipitate in the grooves 37a while agglutinating. It is preferable to uniformly form the grooves 37a on the entire bottom surface not to cause a variation in size of agglutination clumps of the fat-derived cells A during this process.

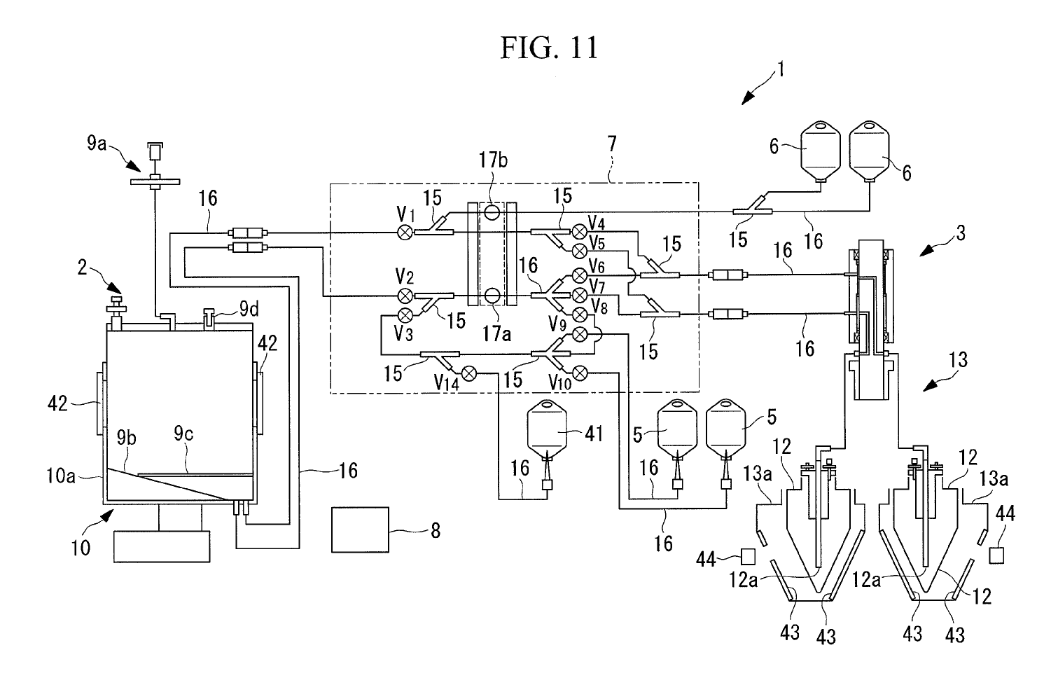

A fourth embodiment of the present invention will be described below with reference to FIG. 11.

Also in this embodiment, the same configurations as those in the first to third embodiments are designated with the same reference numerals, descriptions thereof are omitted, and differences from the first to third embodiments will be mainly described.

As shown in FIG. 11, the cell separation apparatus 1 according to this embodiment includes a reagent bag (cell-stimulating portion, activating-agent supplying portion) 41 containing an activating agent, an interface sensor (state-quantity measuring portion) 42 provided on the outer side of the collecting container 9, heaters 43 provided on the baskets 13a, and temperature sensors 44 measuring the temperature of the centrifuge containers 12. The interface sensor 42, the heaters 43, and the temperature sensors 44 may have the same configurations as those of the interface sensor 33, the heater 30, and the temperature sensor 31 of the second embodiment.

The reagent bag 41 is connected to the centrifuge containers 12 through the conveyance path system 7. The reagent bag 41 contains an activating agent solution having a concentration higher than that in use. As the activating agent, a growth factor or a NO-inducing factor is used.

As the growth factor, for example, bFGF, HGF, or SDF-1 is used. As the NO-inducing factor, an endothelial NO synthase is used. In addition, a mitochondrial potassium channel opener, an ACE inhibitor, a tatin, an angiotensin-converting enzyme inhibitor, an angiotensin II type 1 receptor blocker, a PPAR-gamma agonist, erythropoietin, haeme oxygenase, sphingosine-1-phosphate, FTY720, PPAR-gamma, or agonist pioglitazone can be used.

The computer 8, after digestion of fat tissue, but before sending a cell suspension from the collecting container 9 to the centrifuge containers 12, measures the amount of cell suspension by detecting the surface of the cell suspension in the collecting container 9 using the interface sensor 42. Furthermore, the computer 8 calculates the supply amount of the activating agent solution based on the amount of the measured cell suspension so that the final concentration of the activating agent when the activating agent solution is supplied to the cell suspension becomes a predetermined value, and supplies the calculated amount of the activating agent solution to the centrifuge containers 12. The computer 8 controls the temperature of the heaters 43 so that the temperature measured by the temperature sensors 44 is maintained at a predetermined value. It is preferable that the set temperature value of the temperature sensors 44 be appropriately selected according to the activating agent to be used so that the activating agent most efficiently acts on fat-derived cells.

The effects of the thus-configured cell separation apparatus 1 will be described below.

The cell separation apparatus 1 according to this embodiment produces a cell suspension in the collecting container 9, measures the amount of cell suspension, and then conveys the cell suspension to the centrifuge containers 12. Subsequently, the cell separation apparatus 1 supplies an activating agent solution to the centrifuge containers 12 so that the concentration of the activating agent becomes a predetermined level. Then, after waiting for a predetermined time for sufficiently performing the reaction between the fat-derived cells and the activating agent, the cell separation apparatus 1 removes the activating agent together with the digestive enzyme solution by washing the fat-derived cells.

Thus, according to this embodiment, also adding an activating agent to fat-derived cells provides advantages in that the fat-derived cells are activated with a simple configuration and operation and that the effectiveness of subsequently performed therapy can be improved. Furthermore, an advantage is provided in that the fat-derived cells can be efficiently activated by controlling the supply amount of the activating agent solution according to the amount of cell suspension and by reacting the fat-derived cells with the activating agent at a predetermined temperature.

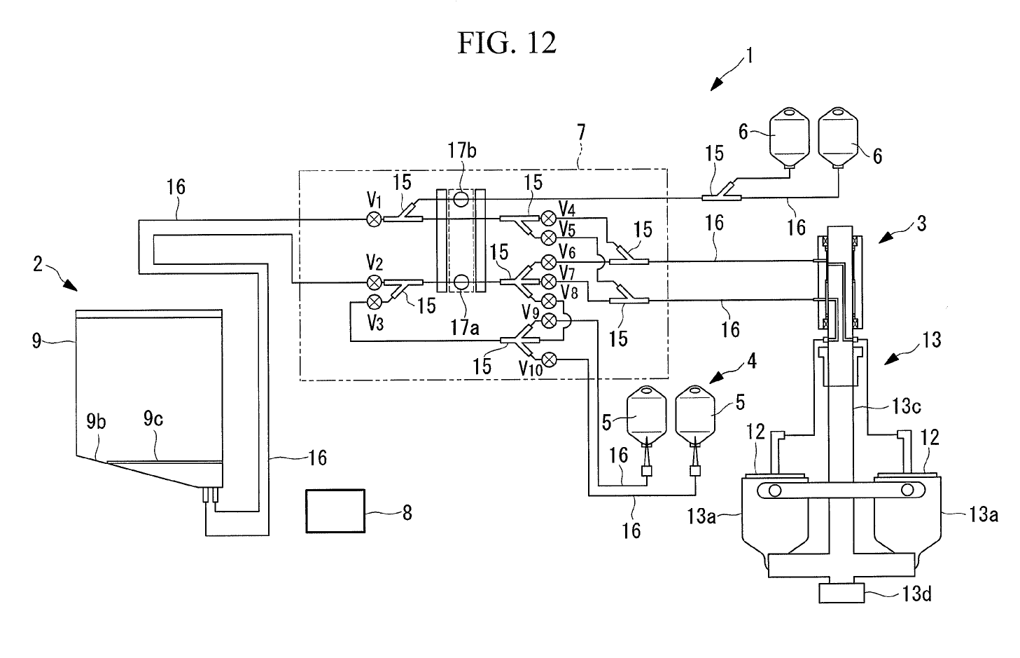

A cell separation apparatus 1 according to a fifth embodiment of the present invention will be described with reference to FIGS. 12 to 18.

FIG. 12 is an overall configuration diagram of the cell separation apparatus 1 according to this embodiment.

The cell separation apparatus 1 according to this embodiment differs from the above-described embodiments in that the cell concentrating unit 3 has a cell-counting portion 51 counting the number of fat-derived cells in the centrifuge containers 12 supported by the centrifuge 13, as described below, and a cell-stimulating portion (efficiency-increasing portion) 52 stimulating fat-derived cells.

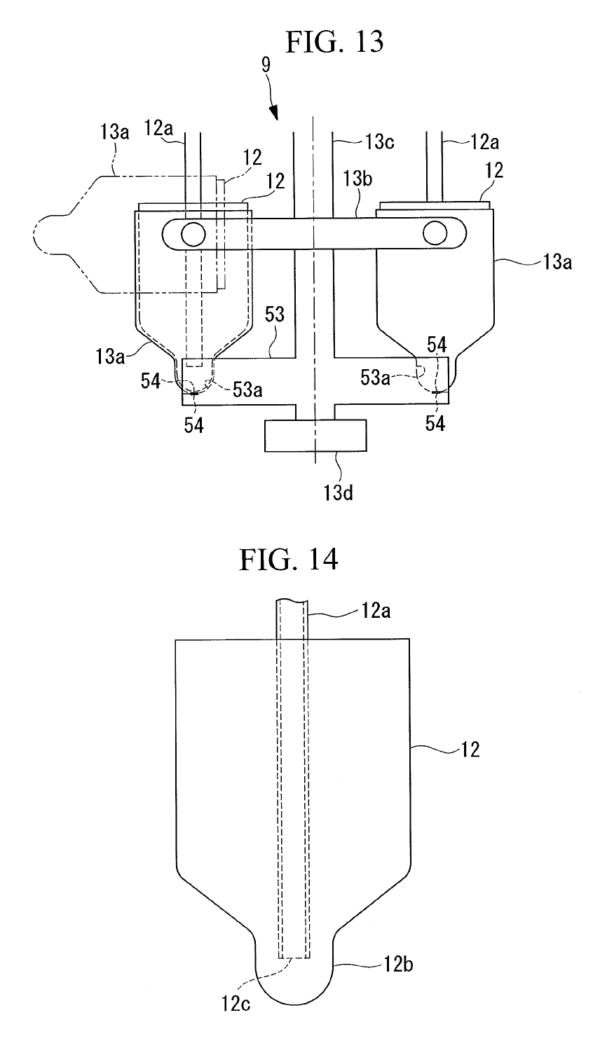

As shown in FIG. 13, the centrifuge 13 includes a shaft 13c having an arm 13b and a motor (rotating portion) 13d rotating the shaft 13c around its central axis. The baskets 13a are supported at both ends of the arm 13b. The shaft 13c is integrally provided with a receiving portion 53 for receiving the bottom portions of the baskets 13a.

The shaft 13c is disposed approximately upright in the vertical direction. The baskets 13a are slightly larger than the centrifuge containers 12 and have approximately the same shapes as those of the centrifuge containers 12. The baskets 13a are supported by both ends of the arm 13b, which is fixed to the shaft 13c approximately orthogonally, so as to be swingable in the direction in which the bottom portions move away from the shaft 13c. By doing so, when the shaft 13c is rotated in the state that the centrifuge containers 12 containing a cell suspension are placed in the baskets 13a, the baskets 13a and the centrifuge containers 12 swing so that the bottom portions are positioned outward in the radial direction. As a result, the fat-derived cells in the cell suspension are agglutinated at the bottom portions of the pockets 12b described below.

As shown in FIG. 14, pipe 12a connected to the conveyance path system 7 is inserted in the centrifuge container 12, and a cell suspension is supplied to the centrifuge container 12 through the pipe 12a. The pipe 12a is connected to the conveyance path system 7 via a rotary joint (not shown) so as not to be twisted during centrifugation.

The bottom portions of the centrifuge containers 12 are each provided with the pocket 12b having an approximately hemispherical shape formed by making a dent on the inside surface. The suction and discharge opening 12c opened at the end of the pipe 12a is arranged at a position away from the bottom of the pocket 12b. By doing so, when the supernatant is discharged from the suction and discharge opening 12c after the centrifugation of a cell suspension, only the supernatant is discharged so that the cell clump precipitated to the bottom of the pocket 12b is not discharged; and when a washing solution is supplied from the suction and discharge opening 12c after discharging the supernatant, the cell clump is efficiently stirred in the washing solution by the force of the inflow of the washing solution. Furthermore, the cell suspension can be stirred by pipetting by repeating suction and discharge from the suction and discharge opening 12c.

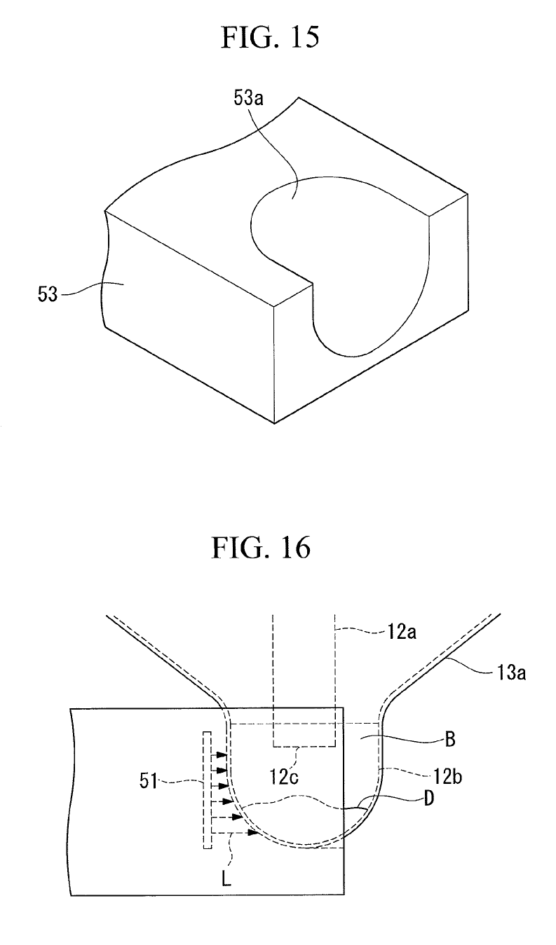

The receiving portion 53 is provided with a receiving face 53a at a position adjacent to the bottom portion of the basket 13a in the state where the rotation of the shaft 13c is stopped. As shown in FIG. 15, the receiving face 53a has an approximately hemispherical shape opening outward in the radial direction with respect to the shaft 13c and has a shape complementary to the bottom portion of the basket 13a. By doing so, when starting and stopping centrifugation, the basket 13a can smoothly move away from the receiving face 53a and move close to the receiving face 53a without being prevented from swinging.

On this occasion, the bottom portion of the basket 13a and the receiving face 53a are provided with magnets 54 generating attractive force so that the bottom portion of the basket 13a descending after completion of centrifugation reliably returns to the position corresponding to the receiving face 53a. The magnets 54 have a magnet force sufficiently smaller than the centrifugal force acting on the bottom portion of the basket 13a during the centrifugation. The magnets 54 may be electromagnets that start to operate for generating magnetic force at the end of centrifugation.

The cell-counting portion 51 and the cell-stimulating portion 52 are both provided on the receiving face 53a.

As shown in FIG. 16, the cell-counting portion 51 emits detection light L toward the pocket 12b received by the receiving face 53a in approximately the horizontal direction and detects feedback light, which is the detection light L reflected or scattered backward. Here, when the cell-counting portion 51 detects feedback light in the state where a cell clump D has precipitated at the bottom portion of the pocket 12b, the intensity of feedback light changes at the interface between the cell clump D and supernatant B. The cell-counting portion 51 detects the position of the upper end of the cell clump D from this change in intensity of the feedback light, calculates the volume of the cell clump D from the detected upper end position, and calculates the number of fat-derived cells from the calculated volume.

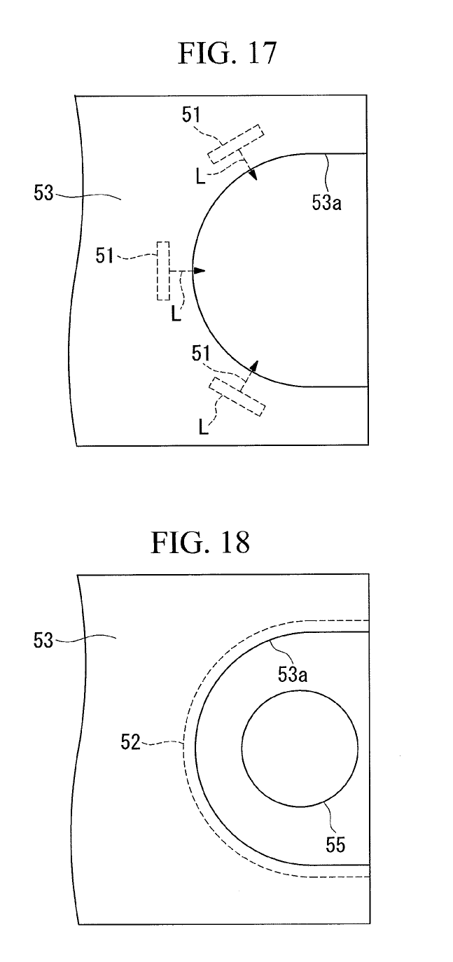

On this occasion, as shown in FIG. 17, the detection light L is preferably emitted from at least three positions so that the position of the upper end of the cell clump D is detected from at least three directions. By doing so, the volume of the cell clump D is more precisely determined by three-dimensionally detecting the shape of the upper end face of the cell clump D, and the number of fat-derived cells contained in the cell clump D can be correctly calculated.

The bottom portions of the baskets 13a are provided with windows (not shown) for transmitting the detection light L and feedback light at positions where the light passes through so that detection light L and feedback light are not obstructed on the way between the cell-counting portion 51 and the centrifuge container 12 disposed in the basket 13a. Alternatively, the bottom portions of the baskets 13a may be provided with openings so that the bottom portions of the centrifuge containers 12 can be seen through the baskets when the centrifuge containers 12 are placed in the baskets 13a.

As shown in FIG. 18, the cell-stimulating portion 52 is a heater (hereinafter, also referred to as heater 52) provided on the receiving face 53a. The receiving face 53a is also provided with a temperature sensor 55. By doing so, the inside of the pocket 12b received by the receiving face 53a is efficiently heated, and the temperature of the inside of the pocket 12b is more precisely detected. Temperature data detected by the temperature sensor 55 is sent to the computer 8, and the temperature of the heater 52 is controlled by the computer 8 to be kept constant at a predetermined temperature.

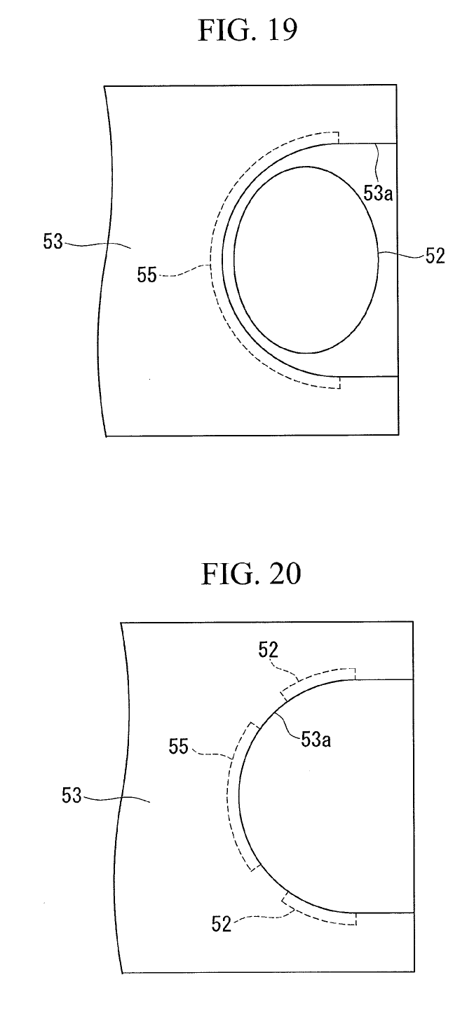

The arrangement of the heater 52 and the temperature sensor 55 shown in FIG. 18 is merely an example and can be suitably modified. For example, as shown in FIG. 19, the heater 52 may be disposed on the bottom, and the temperature sensor 55 may be disposed on the side face. Alternatively, as shown in FIG. 20, both the heater 52 and the temperature sensor 55 may be disposed on the side face.

After completion of the last centrifugation and discharge of supernatant in the cell concentrating unit 3, the computer 8 lets the cell-counting portion 51 calculate the number of fat-derived cells. The computer 8 determines the length of time for the subsequent heat treatment based on the number of fat-derived cells measured by the cell-counting portion 51 and adjusts the temperature of the heater 52 so that the temperature detected by the temperature sensor 55 is maintained at the set temperature for the determined treatment time. Here, the set temperature is a temperature that can appropriately stimulate the fat-derived cells, specifically, 38 to 42° C. and preferably 40° C.

The computer 8 controls the pipes 12a to repeat suction and discharge slowly during the heat treatment so that the cell concentrate, being a mixture of fat-derived cells and supernatant, in the centrifuge containers 12 is subjected to pipetting.

The effects of the thus-configured cell separation apparatus 1 will be described below.

The cell separation apparatus 1 according to this embodiment centrifuges the cell suspension produced in the decomposition treatment unit 2 with the cell concentrating unit 3 and then washes the fat-derived cells by repeated discharge of supernatant, supply of a washing solution, and centrifugation. After washing the fat-derived cells, the cell separation apparatus 1 counts the number of fat-derived cells precipitated in the pocket 12b and determines the length of time for heat treatment. Subsequently, the cell separation apparatus 1 increases the temperature of the heater 52 and then continues pipetting the fat-derived cells and a small amount of supernatant in the pocket 12b for the determined treatment time while maintaining the temperature of the pocket 12b at the set temperature, preferably, 40° C.

By the procedure above, a cell concentrate in which fat-derived cells are suspended in a small amount of the washing solution is obtained in the pocket 12b as a final product.

Thus, according to this embodiment, the fat-derived cells are activated by being stimulated by heat treatment immediately before the completion of the entire process. By doing so, the efficiency of fat-derived cells having high activity just after activation is increased, which provides an advantage in that the therapeutic effect of the final product can be improved. By performing pipetting during heat treatment, heat can be uniformly transferred to the fat-derived cells in the cell concentrate, which provides an advantage in that the fat-derived cells are efficiently activated.

Since such activation of fat-derived cells can be achieved merely by heating the insides of the centrifuge containers 12 from the outsides with the heaters 52, the sterility of the final product can be guaranteed. Furthermore, the number of treatments, such as centrifugation, is not increased, which provides an advantage in that fat-derived cells can be recovered in a state where they retain their healthiness.

Furthermore, in conventional cell separation apparatuses, since the positions of agglutinated cells in centrifuge containers are unclear, stimulation may be applied to positions where the cells are not agglutinated, which causes a reduction in activation efficiency. To address this problem, in the cell separation apparatus 1 according to this embodiment, the cell-counting portion 51 is provided to detect the position of agglutinated cells, and thereby stimulation can be reliably applied to the cells to improve the activation efficiency.

In this embodiment, stimulation of fat-derived cells is performed by heating. Instead of this, fat-derived cells may be stimulated by irradiation with ultrasound or infrared light.

In such a case, as shown in FIG. 21, an ultrasonic transducer or infrared light source 56 is provided on the receiving face 53a, instead of the heater 52. By doing so, the fat-derived cells in a cell concentrate C can be simply and effectively stimulated and, thereby, can be activated.

On this occasion, the computer 8 adjusts the intensity or output time of the ultrasound or infrared light output from the ultrasonic transducer or infrared light source 56 so as to be proportional to the number of fat-derived cells counted by the cell-counting portion 51. The computer 8 temporarily stops the irradiation of ultrasound or infrared light when the temperature detected by the temperature sensor 55 is higher than a predetermined threshold level and restarts the irradiation of ultrasound or infrared light when the temperature detected by the temperature sensor 55 is lower than a predetermined threshold level. By doing so, the cell concentrate C can be prevented from being excessively increased in temperature by irradiation with ultrasound or infrared light.

Alternatively, by providing a liquid-amount measuring portion (not shown) for measuring the amount of cell suspension in the pocket 12b, the computer 8 may control the ultrasound or infrared light irradiation conditions based on the amount of the cell suspension measured by the liquid-amount measuring portion. As the liquid-amount measuring portion, for example, an optical sensor detecting the position of the surface of a cell suspension or a scale measuring the weight of a cell suspension can be used.

Furthermore, when the ultrasonic transducer is employed as the cell-stimulating portion 52, an optical sensor (not shown) may be provided for detecting the density of fat-derived cells at a plurality of positions of a cell concentrate C or detecting the presence or absence of bubbles in the cell concentrate. By doing so, it is determined whether the cell concentrate C is irradiated with an excessive intensity of ultrasound or not, and the fat-derived cells are prevented from having excessive stimulation applied thereto.

In this embodiment, the cell-stimulating portion 52 is employed as a efficiency-increasing portion, but the efficiency-increasing portion may be configured so as to remove unnecessary cells from the cell suspension. For example, the efficiency-increasing portion may be beads having surfaces on which an antibody that specifically binds to cells other than fat-derived cells in the cell suspension is immobilized. In this case, for example, the computer 8 adjusts the amount of beads to be supplied to the centrifuge containers 12 based on the amount of the cell suspension.

In this embodiment, the cell-stimulating portion 52 is integrally provided on the shaft 13c so as to cover the pocket 12b, but the arrangement of the cell-stimulating portion 52 is not limited to this, as long as the cell concentrate in the pocket 12b can be efficiently stimulated.

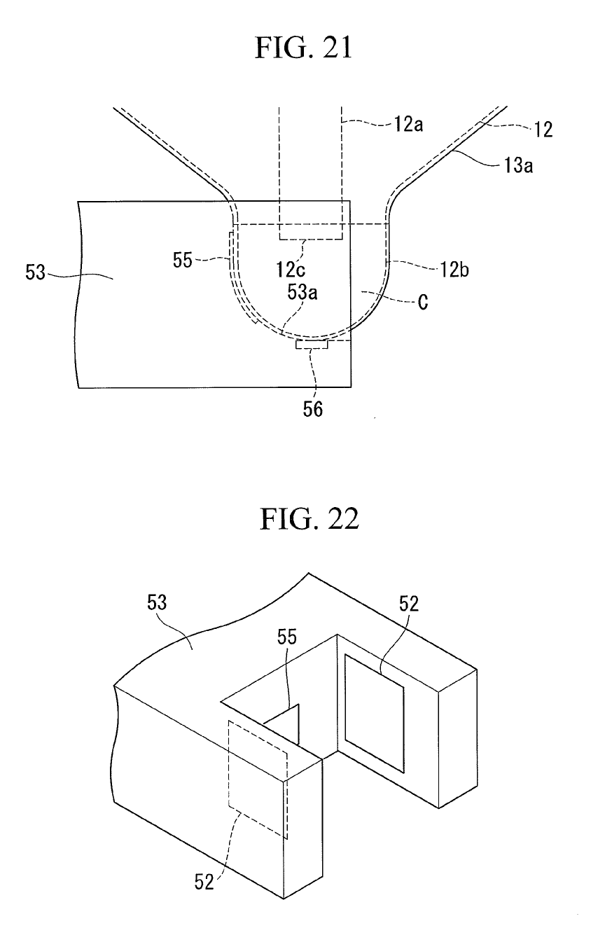

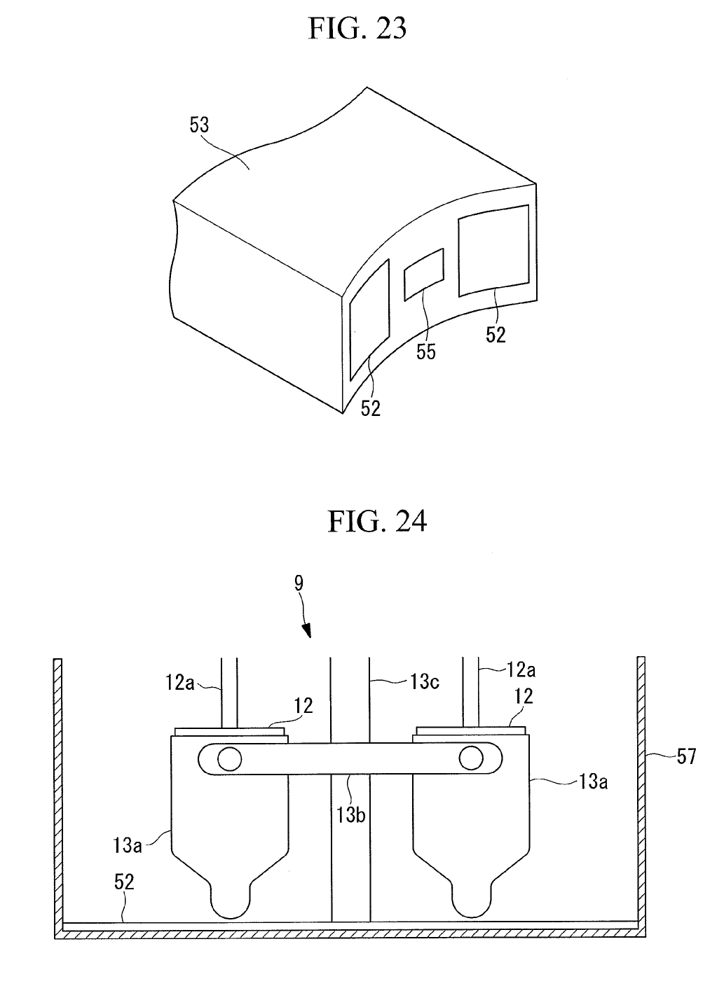

For example, as shown in FIG. 22, the receiving portion 53 may be formed into a U-shape provided with a groove into which the pocket 12b fits, or as shown in FIG. 23, the receiving portion 53 may be formed so as to have a gradually curved face to which the pocket 12b is adjacent. Furthermore, as shown in FIG. 24, by providing a panel 57 surrounding the centrifuge 13, the cell-stimulating portion 52 may be disposed on the entire bottom of the panel 57, separated from the arm 13b and the shaft 13c.

In this embodiment, the cell-counting portion 51 counts the number of fat-derived cells based on the volume of the precipitated cell clump D. Instead of this, the number of fat-derived cells may be counted based on the intensity of light scattered by the cell concentrate C.

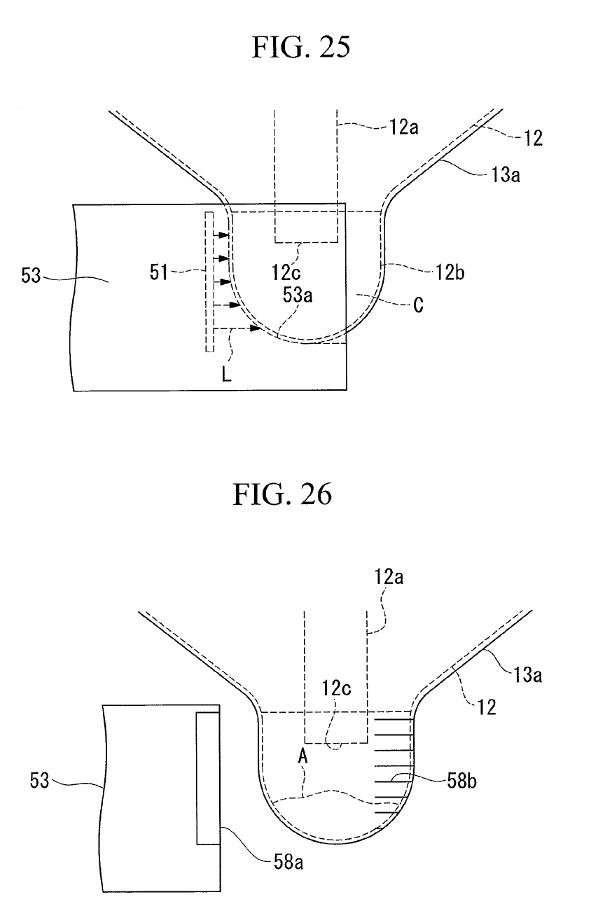

In such a case, fat-derived cells are washed, the supernatant is discharged, the fat-derived cells and a small amount of the supernatant are suspended by pipetting, and then, as shown in FIG. 25, the cell-counting portion 51 emits detection light L′ toward the pocket 12b from the receiving face 53a and detects scattered light of the detection light L′. The intensity of the scattered light at this time is proportional to the density of fat-derived cells in the cell concentrate C. Therefore, the cell-counting portion 51 can count the number of the fat-derived cells based on the intensity of the detected scattered light.

The configuration of the cell-counting portion 51 employed in this embodiment is merely an example and is not limited thereto. For example, as shown in FIG. 26, the cell-counting portion 51 may be composed of a camera 58a provided on the receiving face 53a and a memory 58b provided on the basket 13a, the memory 58b may measure the height position of the cell clump D in an image taken by the camera 58a, and the number of the cells may be calculated from the measured height position. During this process, a part of the basket 13a can be appropriately made of a transparent material or may be provided with a hole so that the camera 58a can photograph the insides of the centrifuge container 12 and the memory 58b.

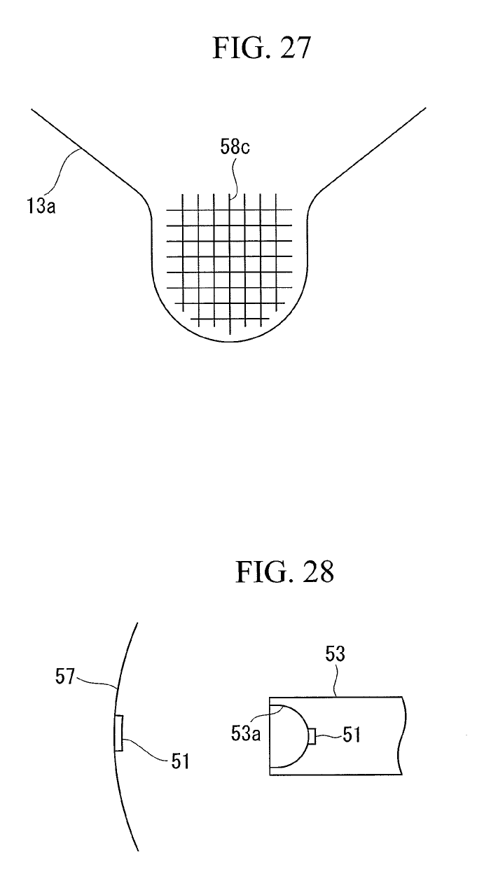

Alternatively, as shown in FIG. 27, grid-like lines 58c provided on the basket 13a may be photographed by the camera 58a in the state where the fat-derived cells are suspended. In this case, since the visibility of the lines 58c varies depending on the concentration of the cell suspension, the number of fat-derived cells can be counted.

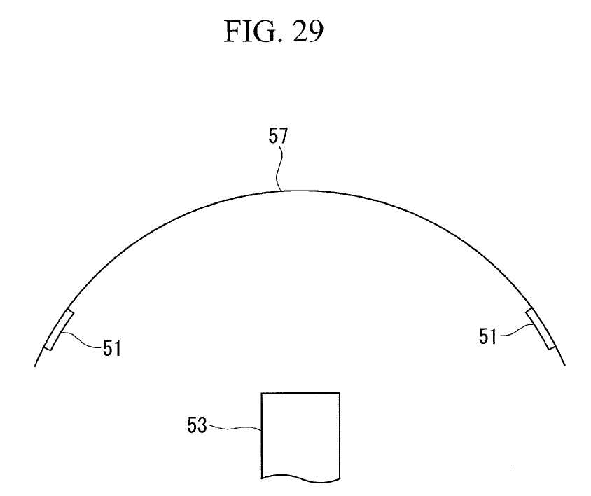

In addition, the cell-counting portion 51, as shown in FIG. 28, may be provided on each of the panel 57 and the receiving face 53a, or, as shown in FIG. 29, two cell-counting portions 51 may be provided on the panel 57. In the case in which the cell-counting portion 51 is provided on the panel 57, the cell-counting portion 51 and the centrifuge container 12 may be misaligned when the rotation of the centrifuge 13 is stopped. Therefore, the motor 13d may be provided with a brake so that the arm 13b stops at a predetermined position with respect to the cell-counting portion 51 when it stops the rotation, or the stop position may be adjusted by rotating the arm 13b in reverse.

In this embodiment, the arrangement of the cell-stimulating portion 52 and the cell-counting portion 51 can be appropriately changed. Specifically, both the cell-stimulating portion 52 and the cell-counting portion 51 may be integrally provided on the arm 13b. Alternatively, the cell-stimulating portion 52 may be integrally provided on the arm 13b, and the cell-counting portion 51 may be provided on the panel 57. Furthermore, the cell-stimulating portion 52 may be provided on the panel 57, and the cell-counting portion 51 may be integrally provided on the arm 13b.

As the cell-counting portion 51, a flow cytometer, a particle size distribution analyzer, or a turbidimeter can be used.

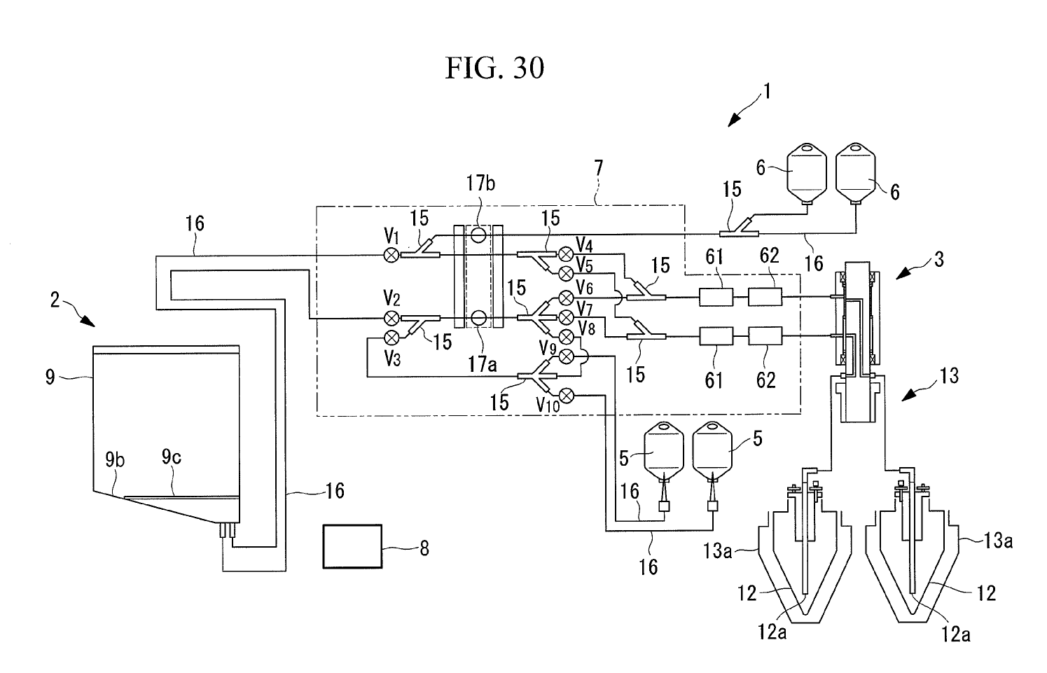

A cell separation apparatus 1 according to a sixth embodiment of the present invention will be described with reference to FIGS. 30 to 34. The same configurations as those in the first to fifth embodiments are designated with the same reference numerals, and descriptions thereof are omitted.

As shown in FIG. 30, the cell separation apparatus 1 according to this embodiment includes cell-counting portions 61 and cell-stimulating portions (cell-treating portion) 62 disposed at intermediates points in the conveyance path system 7.

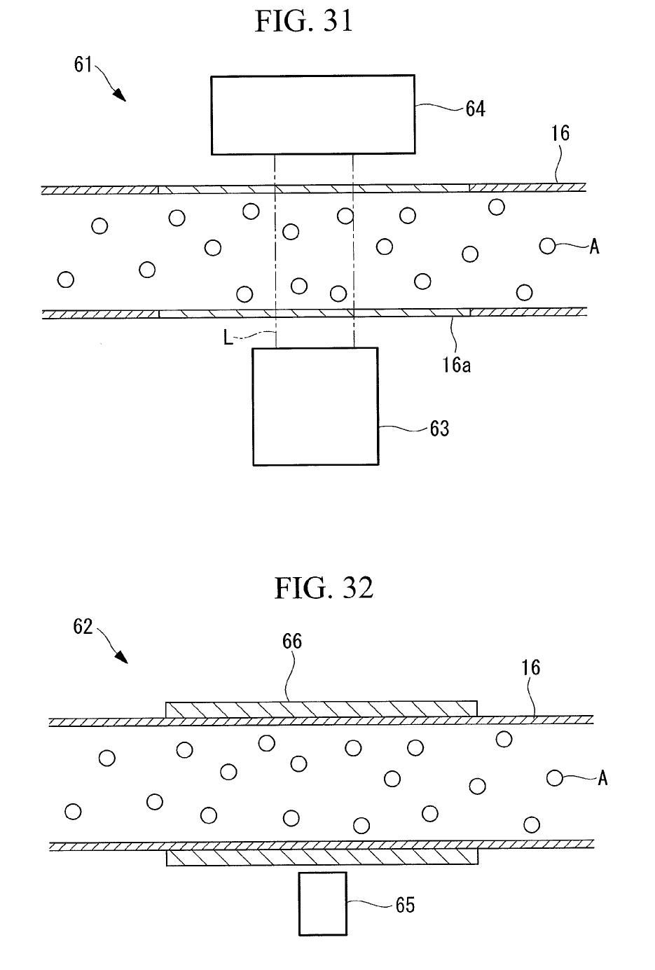

The cell-counting portion 61 is disposed in the tube 16 conveying a cell suspension from the collecting container 9 to the centrifuge containers 12. As shown in FIG. 31, the cell-counting portion 61 includes a laser light source 63 and a detector 64 arranged so as to face each other with the tube 16 therebetween in the radial direction. The laser light L emitted from the laser light source 63 is scattered by the fat-derived cells A contained in the cell suspension in the tube 16 when the light L passes through the tube 16. The intensity of scattered light generated at this time is proportional to the number of fat-derived cells A.

The detector 64 detects the scattered laser light L and calculates the number of fat-derived cells A in the cell suspension based on the intensity of the detected scattered light. Part of the tube 16 may be made of a glass tube 16a at the position interposed between the laser light source 63 and the detector 64 for precisely detecting the scattered light with the detector 64.

The cell-stimulating portion 62 is disposed in the tube 16 conveying a cell suspension from the collecting container 9 to the centrifuge containers 12. As shown in FIG. 32, the cell-stimulating portion 62 includes an ultrasonic transducer 65 generating ultrasound vibrations in the tube 16. Part of the tube 16 is covered with a metal pipe 66, and the ultrasound generated by the ultrasonic transducer 65 is efficiently transmitted to the cell suspension in the tube 16 by the metal pipe 66.

The ultrasound generated in the cell suspension by the ultrasonic transducer 65 is set using various parameters to stimulate the fat-derived cells A while avoiding excessive influence on the fat-derived cells A. For example, the ultrasound output from the ultrasonic transducer 65 preferably has a frequency of about 1.5 MHz, a burst width of about 200 μs, a repetition frequency of about 1 kHz, an effective ultrasound radiation area of about 3.88 cm², and an output power of about 117 mW. With this, the fat-derived cells A in the cell suspension are appropriately stimulated by the ultrasound, and the function of the fat-derived cells, which has been decreased, is activated.

The computer 8 controls the laser light source 63 to emit laser light L when conveyance of a cell suspension from the decomposition treatment unit 2 to the cell concentrating unit 3 is started. The computer 8 receives, from the detector 64, information on the number of fat-derived cells calculated by the detector 64 and adjusts the output level of the ultrasonic transducer 65 so as to be proportional to the number of fat-derived cells.

The positions at which the cell-counting portion 61 and the cell-stimulating portion 62 are disposed are not limited to the positions shown in FIG. 30. The cell-counting portion 61 is preferably disposed at the stage preceding the cell-stimulating portion 62. By doing so, the measurement result obtained by the cell-counting portion 61 can be effectively reflected in the control of the cell-stimulating portion 62 by the computer 8. Furthermore, the cell-stimulating portion 62 is preferably disposed just before the cell concentrating unit 3. By doing so, the conveyance time of a cell suspension from the cell-stimulating portion 62 to the cell concentrating unit 3 is shortened, and thereby the activity of the fat-derived cells at the time of completion of treatment in the cell concentrating unit 3 can be maintained in a higher state.

The operation of the thus-configured cell separation apparatus 1 will be described.

The cell separation apparatus 1 according to this embodiment conveys the cell suspension produced in the collecting container 9 toward the centrifuge containers 12 and, at the same time, starts the emission of laser light L from the laser light source 63 and the output level of ultrasound from the ultrasonic transducer 65. After completion of the conveyance of the cell suspension, the cell separation apparatus 1 washes the fat-derived cells in the cell concentrating unit 3 to obtain a concentrate of the fat-derived cells as a final product.

In this case, according to this embodiment, the fat-derived cells are activated by irradiation with the ultrasound during conveyance to the centrifuge containers 12. With this, a final product rich in fat-derived cells in an activated state effective for therapy can be recovered from the centrifuge containers 12, and even if the final product is directly used for therapy, a high therapeutic effect can be obtained. In particular, by adjusting the intensity of stimulation by ultrasound according to the number of fat-derived cells, stimulation of an appropriate magnitude can be applied to the fat-derived cells, which provides an advantage in that the fat-derived cells can be effectively activated.

In addition, even if the cell-stimulating portion 62 is thus-provided, the length of time for the entire treatment and the number of treatment steps, in particular, the number of centrifugation treatments, are not increased, which provides an advantage in that the fat-derived cells can be recovered in a state where their healthiness is maintained. Furthermore, the fat-derived cells are stimulated in the tube 16 in a noncontact manner, which provides an advantage in that the sterility of the fat-derived cells can be guaranteed.

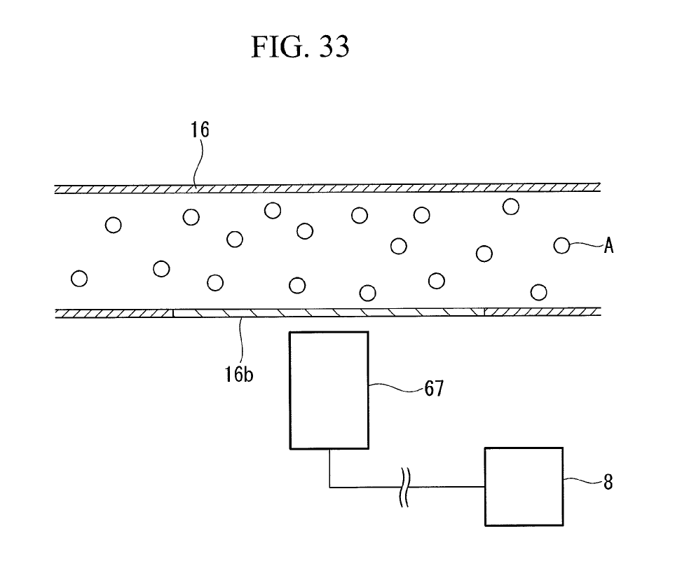

In this embodiment, the cell-counting portion 61 counts the number of fat-derived cells based on the scattered laser light L. Instead of this, as shown in FIG. 33, a photographing portion, for example, a charge coupled device (CCD) camera 67, may be provided for photographing the inside of the tube 16, and the number of fat-derived cells in an image captured by the CCD camera 67 may be counted.

For example, the image captured by the CCD camera 67 is input to the computer 8. The computer 8 counts the number of fat-derived cells present in the image by processing the input image. During this process, part of the tube 16 may be provided with a window 14b made of a material having high transparency, such as glass, so that a clear image of the fat-derived cells can be captured by the CCD camera 67. By doing so, the number of fat-derived cells can be counted without making contact with the fat-derived cells.

The number of fat-derived cells also may be counted by a cell counter or by using a fluorescent reagent that emits fluorescence due to the activity of living cells.

When the cell counter is used, for example, a sample port (not shown) for sampling a part of the cell suspension is disposed in the tube 16, and a small amount of the cell suspension being conveyed from the collecting container 9 to the centrifuge containers 12 is sampled. An operator counts the number of fat-derived cells in the sampled cell suspension under a microscope using the cell counter.

When the fluorescent reagent is used, for example, the fluorescent reagent is added to the collecting container 9 during the treatment in the decomposition treatment unit 2, and the fluorescence intensity of the cell suspension flowing in the tube 16 is measured by a fluorometer (not shown) while being conveyed to the centrifuge containers 12. The measured fluorescence intensity is proportional to the number of fat-derived cells.

By doing so, the number of fat-derived cells can be counted while maintaining the sterility of the fat-derived cells.

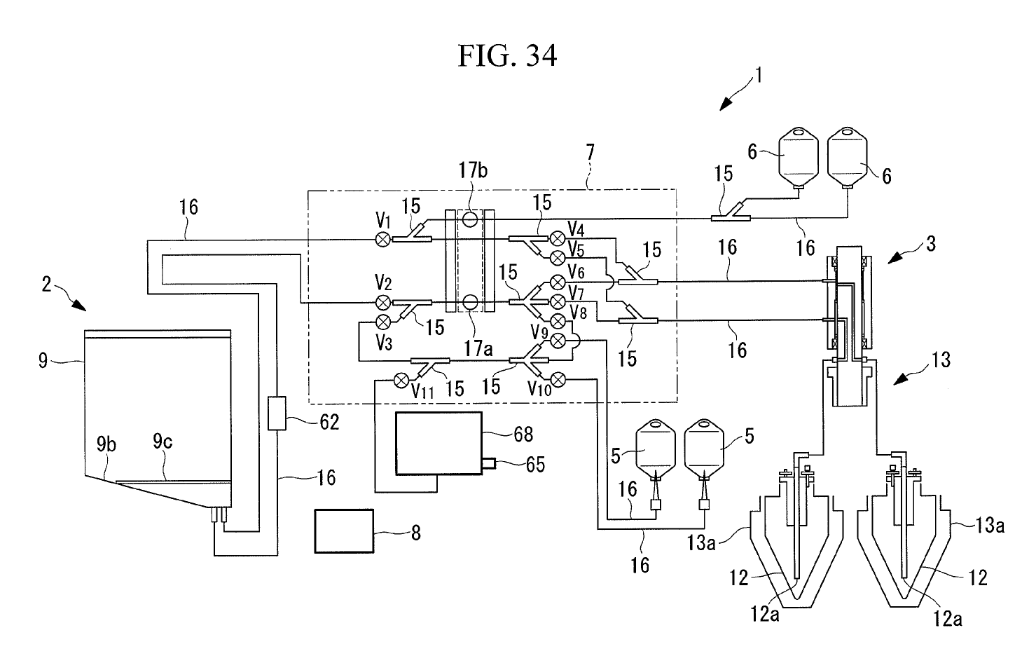

In this embodiment, the cell suspension flowing in the tube 16 is irradiated with ultrasound. Instead of this, as shown in FIG. 34, a treatment container 68 for temporarily storing the cell suspension discharged from the collecting container 9 may be provided, and the cell suspension stored in the treatment container 68 may be irradiated with ultrasound.

By doing so, a large number of fat-derived cells can be collectively activated by a single irradiation with ultrasound.

In this embodiment, a light source (cell-treating portion) may be provided instead of the ultrasonic transducer 65, and the fat-derived cells may be activated by irradiating the inside of the tube 16 with light, for example, infrared or ultraviolet light.

The cell suspension flowing in the tube 16 is irradiated with light at a sufficiently low output that does not damage the fat-derived cells. The computer 8 adjusts the intensity of the light so as to be proportional to the number of fat-derived cells counted by the cell-counting portion 61. By doing so, the fat-derived cells can be activated in a noncontact manner, using a simple configuration and operation.

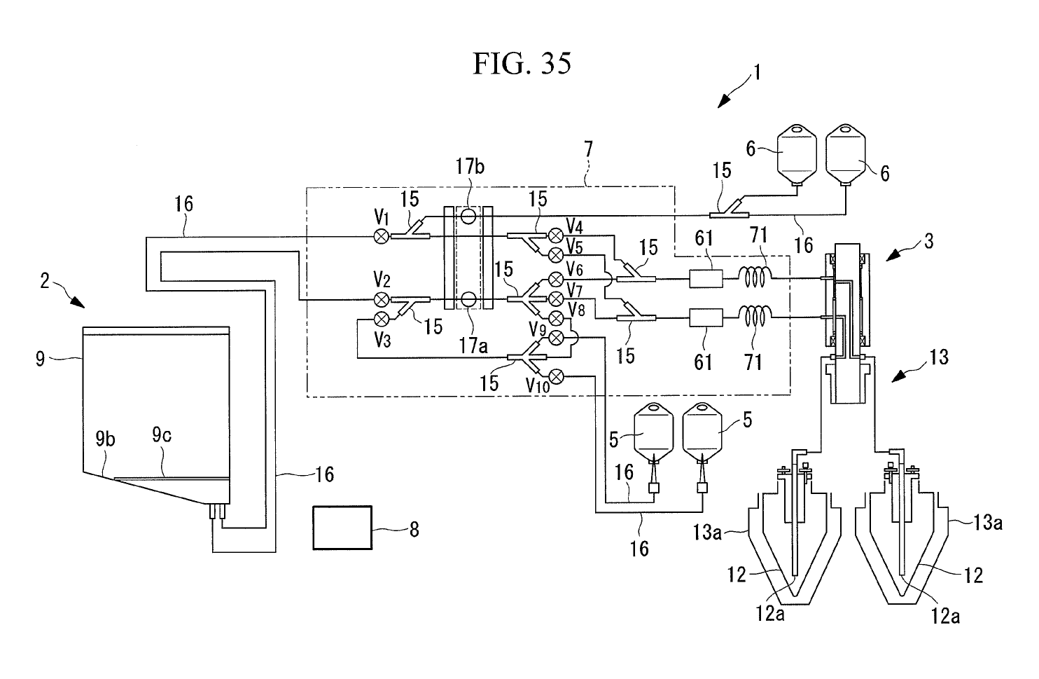

A cell separation apparatus 1 according to a seventh embodiment of the present invention will be described with reference to FIGS. 35 to 38. In this embodiment, the same configurations as those in the first and sixth embodiments are designated with the same reference numerals, and descriptions thereof are omitted.

As shown in FIG. 35, the cell separation apparatus 1 according to this embodiment differs from that of the sixth embodiment in that it includes a cell-sorting channel (cell-treating portion) 71 instead of the cell-stimulating portion 62.

The cell-sorting channel 71 is composed of a pipe having both ends connected to the tube 16 and an antibody, for example, a CD34 antibody, that specifically binds to cells other than fat-derived cells, such as erythrocytes, wherein the antibody is immobilized on the inner surface of the pipe. The pipe is provided with a long length, while avoiding an increase in the area that it takes up, by, for example, being spirally coiled. By doing so, the contact area between the antibody and the cell suspension is increased so that non-targeted cells other than the fat-derived cells can be reliably captured by the cell-sorting channel 71.

The computer 8 adjusts the flow rate of the cell suspension using the supply pump 17a so as to be inversely proportional to the number of fat-derived cells counted by the cell-counting portion 61. Alternatively, when the number of fat-derived cells counted by the cell-counting portion 61 is larger than a predetermined threshold level, the computer 8 performs control so that the density of the fat-derived cells is maintained at a predetermined threshold level or less by diluting the cell suspension by letting a washing solution flow into the tube 16 from the washing solution bag 5, while adjusting the flow rate.

The operation of the thus-configured cell separation apparatus 1 will be described.

The cell separation apparatus 1 according to this embodiment conveys the cell suspension produced in the collecting container 9 from the collecting container 9 to the centrifuge containers 12. During this process, the fat-derived cells in the cell suspension are sorted by the cell-sorting channel 71 and are conveyed to the centrifuge containers 12.

Thus, according to this embodiment, since a cell suspension from which impurities, such as erythrocytes, are removed is conveyed to the centrifuge containers 12, a final product having highly purified fat-derived cells effective for therapy can be obtained. Thus, when a recovered final product is directly used for therapy, for example, the function of the fat-derived cells is not impeded by impurities such as erythrocytes, which provides an advantage in that the therapeutic effect of the fat-derived cells can be sufficiently shown to improve the therapeutic effect. The closed system of the entire cell separation apparatus 1 can be maintained even when the cell-sorting channel 71 is provided in the tube 16, which provides an advantage in that the sterility of the final product can be guaranteed.

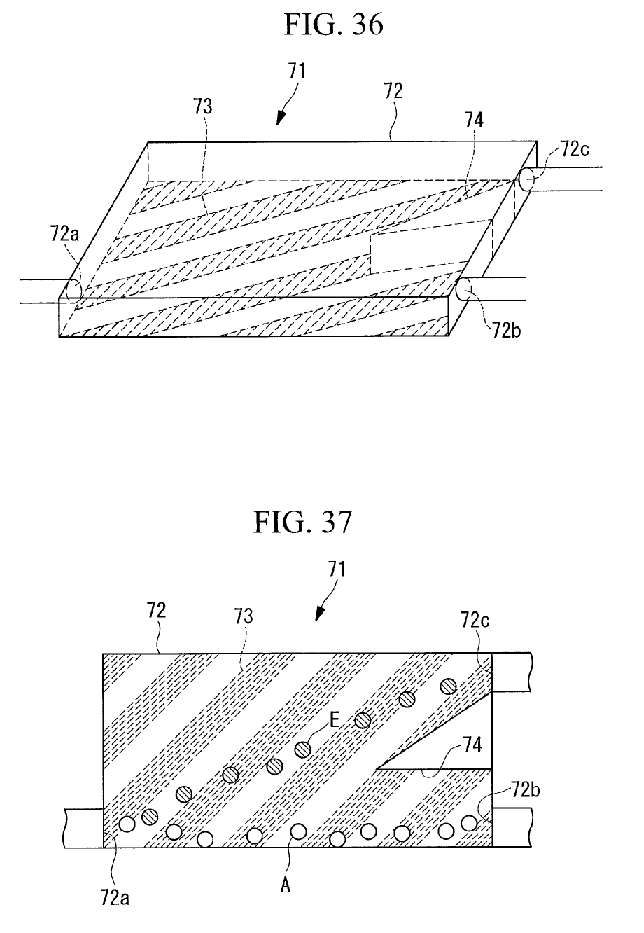

In this embodiment, the cell-sorting channel 71 has a configuration in which an antibody is immobilized on the inner surface of the pipe. Instead of this, as shown in FIG. 36, the cell-sorting channel 71 may have a configuration in which a separation container 72 is arranged in the tube 16 and an antibody immobilized on the bottom of the separation container 72 forms linear adsorption layers 73. The adsorption layers 73 are formed so as to incline, for example, at 45 degrees with respect to the flow direction of a cell suspension and are arranged approximately parallel to one another with gaps therebetween.

In the separation container 72, as shown in FIG. 37, the fat-derived cells A among the cells in a cell suspension being conveyed from the collecting container 9 and entering through the inlet 72a flow in approximately straight line according to the flow direction of the cell suspension and are discharged from the outlet 72b disposed on the concentrating unit side at approximately the front of the inlet 72a. On the other hand, cells E other than the fat-derived cells obliquely flow in the separation container 72 so as to follow the incline of the adsorption layers 73 and are discharged from the outlet 72c disposed on the waste solution side at the diagonal corner with respect to the inlet 72a. In order to more precisely separate the cells to be discharged from the outlets 72b and 72c, a partition 74 may be disposed between the outlets 72b and 72c.

During this process, the flow rate of the cell suspension is adjusted so that the cells A and E in the cell suspension flow along the bottom of the separation container 72 and so that the cells E other than the fat-derived cells flow without adhering to the adsorption layers 73.

By doing so, since the fat-derived cells A are sorted from the cells contained in the cell suspension and are conveyed to the centrifuge containers 12, a final product of highly purified fat-derived cells can be obtained.

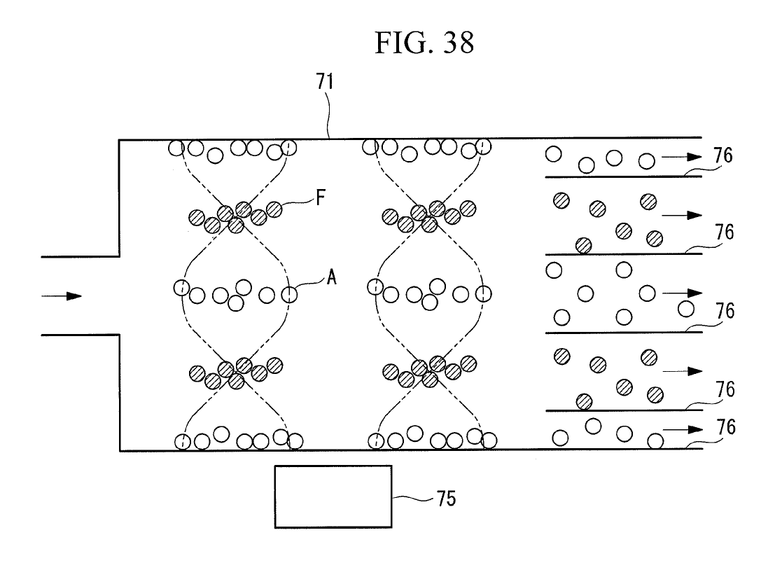

In this embodiment, as shown in FIG. 38, the fat-derived cells may be sorted from the cell suspension using ultrasound in the cell-sorting channel 71.

As shown by broken lines in FIG. 38, an ultrasonic transducer 75 generates ultrasound in the cell suspension in the cell-sorting channel 71 while adjusting the frequency so that standing waves are formed in the direction intersecting, preferably, perpendicular to the flow direction of the cell suspension. By doing so, the fat-derived cells A, having negative acoustical properties, are captured by the ultrasound antinodes, and the erythrocytes F, having positive acoustical properties, are captured by the ultrasound nodes.

Therefore, branching flow paths 76 are formed for allowing the fat-derived cells A captured by the ultrasound antinodes to flow to the centrifuge containers 12 and the erythrocytes F captured by the ultrasound nodes to flow to the waste solution bag 6. By doing so, the fat-derived cells A can be sorted from the cell suspension and conveyed to the centrifuge containers 12. In addition, since the fat-derived cells A are activated by ultrasound in the cell-sorting channel 71, the therapeutic effect of the final product can be further enhanced.

In this embodiment, the cell separation apparatus may include the cell-stimulating portion 62 according to the sixth embodiment. By doing so, the therapeutic effect of the final product can be maximized.

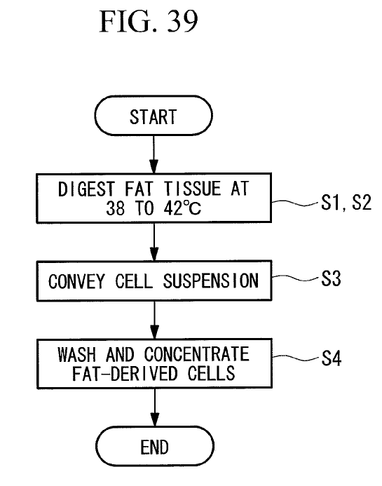

A graft material producing process according to an eighth embodiment of the present invention will be described below with reference to FIGS. 39 to 42.

As shown FIG. 39, the graft material producing process according to this embodiment includes a digestion step S1 for producing a cell suspension by digesting fat tissue and thereby separating fat-derived cells from the fat tissue; an activation step S2 for activating the fat-derived cells by warming the cell suspension at a predetermined temperature; a conveyance step S3 for conveying the cell suspension; and a washing step S4 for washing the fat-derived cells. The activation process of fat-derived cells according to the second embodiment of the present invention corresponds to the activation step S2.

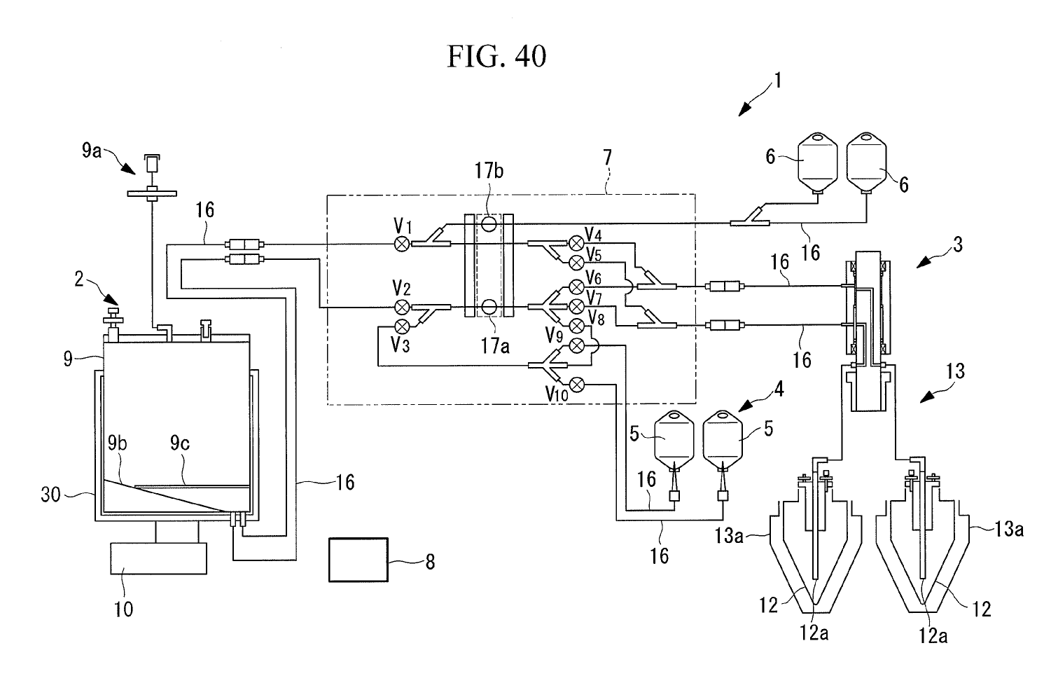

FIG. 40 shows an example of a graft material producing apparatus 100 for implementing the graft material producing process according to this embodiment.

The basic configuration of the graft material producing apparatus 100 is the same as the cell separation apparatus 1 according to the first to seventh embodiments described above. In this embodiment, the same configurations as those in the first to seventh embodiments are designated with the same reference numerals, and descriptions thereof are omitted. The graft material producing apparatus 100 conducts the digestion step S1 using the decomposition treatment unit 2 and conducts the washing step S4 using the cell concentrating unit 3.

The decomposition treatment unit 2 includes a heater 30 warming the inside of the collecting container 9. The heater 30 may be any heater that can warm the inside of the collecting container 9 to at least 40° C. by adjusting the temperature at the outside of the collecting container 9. For example, an air jacket system arranged so as to cover the side faces of the collecting container 9, a liquid circulating system in which a warmed liquid is circulated in, for example, a pipe arranged on the outer surface of the collecting container 9, or heat transfer wiring arranged on the outer surface of the collecting container 9 is used.

The graft material producing process using the thus-configured graft material producing apparatus 100 will be described below.

First, the digestion step S1 is performed by stirring fat tissue and a digestive enzyme solution in the collecting container 9. During this process, the activation step S2 is simultaneously performed by stirring the fat tissue and the digestive enzyme solution, while keeping the temperature inside the collecting container 9 at 38 to 42° C., preferably, at 40° C., with the heater 30. The digestion step S1 and the activation step S2 are performed for at least 10 minutes, more preferably, about 30 minutes. After the digestion step S1 and the activation step S2, the cell suspension produced in the collecting container 9 is conveyed to the centrifuge containers 12 in the conveyance step S3.

Subsequently, in the washing step S4, the cell suspension is centrifuged to precipitate the fat-derived cells, and the supernatant is discharged into the waste solution bag 6 to remove the digestive enzyme from the fat-derived cells. Then, a washing solution is supplied to the centrifuge containers 12 from the washing solution bag 5 through the conveyance path system 7, and the fat-derived cells are washed by centrifuging the washing solution and the fat-derived cells and discharging the supernatant into the waste solution bag 6. If necessary, the washing of the fat-derived cells is repeated several times. By doing so, finally, a precipitated agglomeration of the fat-derived cells with high purity can be obtained in the centrifuge containers 12. Then, by recovering the precipitated agglomeration of the fat-derived cells and a small amount of the supernatant from the centrifuge containers 12, a graft material consisting of a concentrated solution of the fat-derived cells can be produced. The graft material may contain another suitable material in addition to the concentrated solution of the fat-derived cells.

According to the thus-produced graft material, the fat-derived cells are stimulated by being heated at 38 to 40° C. in the activation step S2 to activate their function, which has been decreased, and, thereby, fat-derived cells having an activated function are produced, which provides an advantage in that a sufficient therapeutic effect can be promptly obtained even if the produced graft material is directly used for therapy.

Furthermore, the fat-derived cells can be stimulated without coming into contact with the outside of the collecting container 9, which provides advantages in that the sterility of the fat-derived cells can be guaranteed and that the fat-derived cells can be effectively activated by a simple configuration and operation using just the heater 30. In addition, the time necessary for producing the graft material can be shortened by performing the activation step S2 and the digestion step S1 simultaneously.

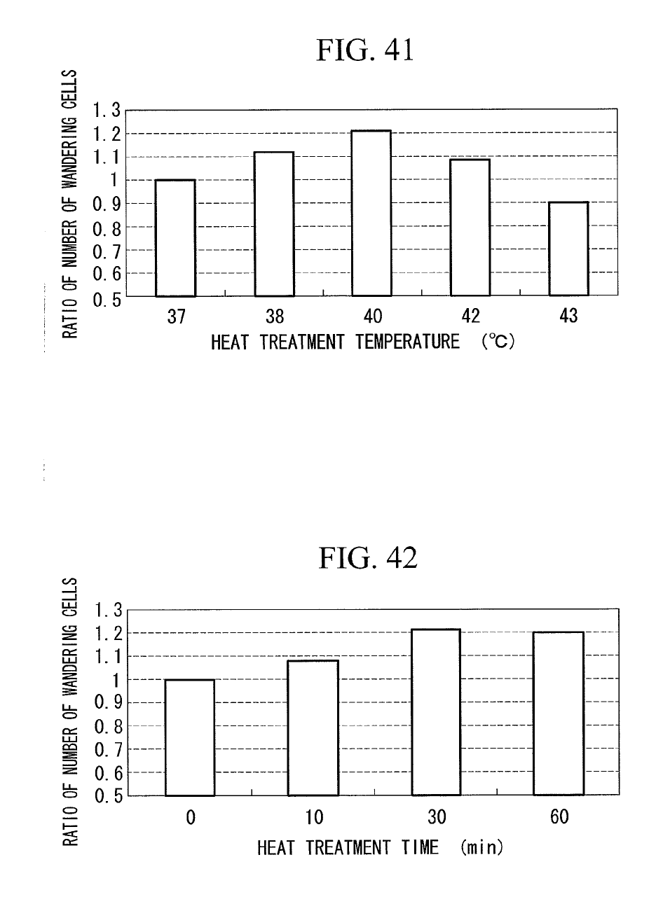

FIGS. 41 and 42 show the experimental results of investigation of the relationship between the temperature of the heat applied to fat-derived cells, or the length of the heat treatment, and its effect on activation of the fat-derived cells.

The experiment was carried out using a 24-well BD BioCoat Matrigel invasion chamber (a product of Becton Dickinson Japan) by the following procedure.

First, EBM-2 (minimal essential medium for endothelial cells), which is a serum-free culture medium, was dispensed in cell culture inserts (hereinafter, referred to as inserts) detached from a companion plate (hereinafter, referred to as plate) to sufficiently hydrate the inserts. Separately, fat-derived cells separated from human fat tissue were subjected to heat treatment at a predetermined temperature for a predetermined time. Then, the cell density of the heat-treated fat-derived cells was adjusted to 1×10⁶ cells/200 mL with X-vivo (culture medium for lymphocytes) to prepare a cell suspension.

The EBM-2 was removed from the inserts, and 250 mL of the cell suspension was dispensed in each insert. Then, 500 μL of an induction medium was added to the plate. As the induction medium, EBM-2 containing 10% fetal bovine serum (FBS) and 100 ng/mL of stromal cell-derived factor (SDF) was used. Then, the inserts were set on the plate and were incubated at 37° C. for 24 hours in a 5% CO₂ environment. Then, the inserts were detached from the plate, the fat-derived cells on the plate were dyed with crystal violet, and the number of the dyed fat-derived cells was counted.

In the experiment above, the number of fat-derived cells (hereinafter, referred to as wandering cells) that migrated to the plate from the inserts by being attracted by FBS and SDF, that is, fat-derived cells having sufficiently high activity, was counted. The effect of activation of the fat-derived cells by heat treatment was evaluated by comparing the number of wandering cells when the fat-derived cells were heated under different conditions.

FIG. 41 is a graph showing the number of wandering cells when fat-derived cells were heated at 37, 38, 40, 42, or 43° C. for 30 minutes, wherein the vertical axis represents the ratio of the number of wandering cells, with the number of wandering cells heated at 37° C. defined as 1. It was confirmed from this experimental result that the number of wandering cells increased with an increase in temperature for heat treatment up to the maximum at 40° C. and then decreased with an increase in temperature. It was thus confirmed that the fat-derived cells are most effectively activated when heated at 40° C., where they show the highest activity.

Then, optimum length of heat treatment time was investigated at 40° C., which was confirmed to be the most effective for activating fat-derived cells. FIG. 42 is showing the number of wandering cells when fat-derived cells were heated at 40° C. for 10, 30, or 60 minutes, wherein the vertical axis represents the ratio of the number of wandering cells, with the number of wandering cells not heated (0 min) defined as 1. This experiment revealed that fat-derived cells start to be activated about 10 minutes after that they have been heated at 40° C. and that a sufficiently high activity can be obtained by being heated for about 30 minutes. Therefore, the length of time for heat treatment of fat-derived cells at 40° C. is preferably 10 minutes or more, and treatment for about 30 minutes is optimum.

In this embodiment, the activation step S2 and the digestion step S1 are simultaneously performed. Instead of this, the activation step S2 may be performed at the same time as the conveyance step S3 or the washing step S4 or may be performed after the digestion step S1 or the washing step S4.

When the activation step S2 is performed at the same time as the conveyance step S3, for example, a heater is disposed in the tube 16 through which a cell suspension passes, and the temperature of the cell suspension is maintained at 38 to 42° C. when the cell suspension flows in the tube 16. In this case, for example, a circulation path may be disposed in the tube 16 for sufficiently ensuring the time for heat treatment of the fat-derived cells.

When the activation step S2 is performed at the same time as the washing step S4, the washing solution in the washing solution bag 5 is heated at 38 to 42° C., and the fat-derived cells are washed with the warmed washing solution.

When the activation step S2 is performed after the digestion step S1, the digestion step S1 is performed, for example, in the state where the temperature inside the collecting container 9 is maintained at 37° C., and then the set temperature of the heater 30 is increased to increase the temperature inside the collecting container 9 to 38 to 42° C. During this process, the activation step S2 is preferably performed while stirring the cell suspension with the shaker 10 so that the temperature of the cell suspension in the collecting container 9 is uniform at every position.

When the activation step S2 is performed after the washing step S4, for example, another heater for warming each centrifuge container 12 is provided, and the temperature inside the centrifuge container 12 is maintained at 38 to 42° C.

By doing so, the fat-derived cells can be activated with a simple configuration and operation, while maintaining the sterility of the fat-derived cells, and the graft material can be more promptly provided with a high therapeutic effect.

When the activation step S2 is performed after the digestion step S1, since the length of time from the activation of fat tissue until its use for therapy is shortened, the therapeutic effect of the graft material can be further improved because the graft material can be used for therapy while maintaining the activity of the fat-derived cells at a higher level.

The present invention includes the following aspects.

The first aspect of the present invention provides a cell separation apparatus including a decomposition treatment unit for producing a cell suspension by digesting living body tissue to release living body-derived cells from the living body tissue; a cell concentrating unit for generating a cell-concentrated solution by concentrating the cell suspension by centrifugation; and a efficiency-increasing mechanism for increasing the efficiency of the living body-derived cells contained in the cell-concentrated solution.

According to the first aspect of the present invention, the cell suspension containing living body-derived cells generated in the decomposition treatment unit is concentrated in the cell concentrating unit, and, thereby, the living body-derived cells can be extracted from the living body tissue. In this case, since a concentrated solution in which the efficiency of the living body-derived cells is increased by the efficiency-increasing portion is recovered as the final product, the therapeutic effect of the final product can be improved.

In the first aspect, the efficiency-increasing mechanism may include a receiving container connected to a conveyance path system, which conveys the cell suspension by being connected to the decomposition treatment unit and the cell concentrating unit, and receiving the cell suspension; a content-measuring portion measuring the content of the receiving container; a medium-supplying mechanism supplying, to the receiving container, a medium for fractionating erythrocytes and the living body-derived cells contained in the cell suspension; a medium supply amount calculator calculating the supply amount of the medium to be supplied to the receiving container by the medium-supplying mechanism based on the content measured by the content-measuring portion; a controller controlling the medium-supplying mechanism to supply the medium based on the supply amount calculated by the medium supply amount calculator; and a recovery mechanism recovering the living body-derived cells fractionated by the medium in the receiving container while separating the living body-derived cells and the erythrocytes.

By doing so, the receiving container temporarily receives the cell suspension on the way from the decomposition treatment unit to the cell concentrating unit, and the weight of the cell suspension is measured by the content-measuring portion. Then, the controller controls the medium-supplying mechanism to supply the medium to the cell suspension in a supply amount calculated by the medium supply amount calculator based on the amount of the cell suspension. By doing so, the living body-derived cells and erythrocytes are fractionated to recover the living body-derived cells separated from the erythrocytes from the receiving container by the recovery mechanism, and a final product in which erythrocytes are sufficiently removed can be obtained. In addition, since the entire treatment is performed in a closed system, the sterility of the final product can be guaranteed.

In the above-described configuration, the medium may be a hemagglutinating agent that agglutinates the erythrocytes. By doing so, the erythrocytes are agglutinated and, thereby, precipitate in the cell suspension, which enables easy fractionation of the living body-derived cells and the erythrocytes.

When the hemagglutinating agent is used as the medium, the decomposition treatment unit may include a collecting container collecting the living body tissue and a stirring mechanism stirring the inside of the collecting container, and the receiving container may be the collecting container. By doing so, the hemagglutinating agent is supplied to the cell suspension produced in the collecting container, that is, the configuration is shared, resulting in simplification of the configuration. In addition, the agglutination of erythrocytes can be accelerated by stirring the inside of the collecting container with the stirring mechanism.

When the hemagglutinating agent is used as the medium, the recovery mechanism may be disposed between the receiving container of the conveyance path system and the cell concentrating unit and may include a filter that captures the agglutinated erythrocytes and allows the living body-derived cells to pass through. By doing so, the living body-derived cells separated from erythrocytes can be recovered in the cell concentrating unit merely by conveying the cell suspension mixed with the hemagglutinating agent.

In the above-described configuration, the medium may be a density-gradient solution having a specific gravity larger than that of the living body-derived cells and smaller than that of the erythrocytes. By doing so, living body-derived cells and erythrocytes can be easily fractionated.

When the recovery mechanism includes the filter, the cell concentrating unit may include a centrifuge container receiving the cell suspension and a centrifuge performing centrifugation using the centrifuge container, and the receiving container may be the centrifuge container. By doing so, the living body-derived cells and the erythrocytes can be rapidly fractionated by centrifuging the cell suspension and the density-gradient solution received in the centrifuge container, and the configuration can be simplified by making shared use of the configuration.

In the above-described configuration, the content-measuring portion may be a scale measuring the weight of the receiving container or may be an interface sensor detecting the height position of the interface of the cell suspension in the receiving container. By doing so, the contents of the cell suspension received in the receiving container can be measured simply.

In the above-described configuration, the cell separation apparatus may include an erythrocyte concentration-measuring portion measuring the concentration of erythrocytes contained in the cell suspension, and the medium supply amount calculator may calculate a supply amount of the medium based on the erythrocyte concentration measured by the erythrocyte concentration-measuring portion and the content measured by the content-measuring portion. By doing so, the supply amount of medium can be more appropriately calculated.

In the above-described configuration, the cell separation apparatus may include a discharge path connected to the bottom of the receiving container and performing discharging from the receiving container, and the recovery mechanism includes an absorbance sensor measuring absorbance inside the receiving container at different positions in the depth direction and a discharge-amount adjuster adjusting the amount of discharging to the discharge path from the receiving container based on the absorbance measured by the absorbance sensor at each position. By doing so, the liquid volume of the erythrocyte layer can be measured by detecting the height position of the interface between the fractionated living body-derived cells and erythrocytes from the difference in absorbance, and the erythrocyte layer, as the lower layer, can be selectively discharged while retaining the living body-derived cell layer in the receiving container.

In the above-described configuration, the cell separation apparatus may further include a washing mechanism washing the living body-derived cells recovered by the recovery mechanism. By doing so, the medium contained in the living body-derived cell layer can be removed.

In the first aspect, the cell separation apparatus may be configured so that the efficiency-increasing mechanism includes a receiving container connected to a conveyance path system, which conveys the cell suspension by being connected to the decomposition treatment unit and the cell concentrating unit, and receiving the cell suspension; a state-quantity measuring portion measuring a state quantity representing the condition of the cell suspension received in the receiving container; a cell-stimulating portion stimulating the living body-derived cells contained in the cell suspension received in the receiving container; and a controller controlling the intensity of the stimulation given to the living body-derived cells by the cell-stimulating portion based on the state quantity measured by the state-quantity measuring portion.

According to this configuration, the state quantity of the cell suspension when it is temporarily received in the receiving container during the treatment is measured by the state-quantity measuring portion. Then, stimulation with an intensity appropriately controlled by the controller is applied to the cell in the cell suspension by the cell-stimulating portion. By doing so, living body-derived cells in an activated state can be obtained by activating the living body-derived cells while guaranteeing the sterility in a closed system.

In the above-described configuration, the state-quantity measuring portion may be a temperature sensor measuring the temperature of the cell suspension in the receiving container; the cell-stimulating portion may include a heater warming the inside of the receiving container; and the controller may control the temperature of the heater based on the temperature of the cell suspension measured by the temperature sensor. By doing so, the living body-derived cells can be activated by heat. In such a case, a stirring mechanism for stirring the inside of the receiving container may be provided. By doing so, the temperature at every position of the cell suspension can be made uniform by heating the cell suspension while stirring it, and thereby each cell can be equally activated.

In the above-described configuration, the state-quantity measuring portion may be a liquid level sensor measuring the amount of cell suspension in the receiving container; the cell-stimulating portion may include an ultrasonic transducer generating ultrasound in the receiving container; and the controller may control the output level of ultrasound to be generated by the ultrasonic transducer based on the amount of the cell suspension measured by the liquid level sensor. By doing so, the living body-derived cells can be activated by ultrasound.

In the above-described configuration, the state-quantity measuring portion may be an oxygen concentration sensor measuring the oxygen concentration of the cell suspension received in the receiving container; the cell-stimulating portion may include a low-oxygen-gas supplying portion supplying a gas with an oxygen concentration lower than that of air to the receiving container; and the controller may control the supply amount of the gas supplied to the receiving container from the low-oxygen-gas supplying portion based on the oxygen concentration measured by the oxygen concentration sensor. By doing so, the living body-derived cells can be activated by reducing the oxygen concentration in the cell suspension.

In the above-described configuration, the state-quantity measuring portion may be a liquid level sensor measuring the amount of the cell suspension received in the receiving container; the cell-stimulating portion may include an activating-agent supplying portion supplying an activating agent that activates the living body-derived cells in the receiving container; and the controller may control the supply amount of the activating agent to be supplied to the receiving container from the activating-agent supplying portion based on the liquid amount measured by the liquid level sensor. By doing so, the living body-derived cells can be activated by the activating agent. In such a case, it is preferable that an activating agent-removing mechanism for removing the activating agent be provided so that unnecessary activating agent can be removed after the activation of the cell. Furthermore, the activating agent is preferably a growth factor or a nitric oxide-inducing factor, so that the cell can be efficiently activated.

In the above-described configuration, the state-quantity measuring portion may be a cell density meter measuring the density of the living body-derived cells contained in the cell suspension in the receiving container; the cell-stimulating portion may stimulate the living body-derived cells in the cell suspension by bringing the living body-derived cells into contact with one another in the receiving container; and the controller may control the supply amount of the cell suspension to be supplied to the receiving container through the conveyance path system based on the density measured by the cell density meter. By doing so, the living body-derived cells can be activated by coming into contact with one another under appropriate conditions.

In the above-described configuration, the cell-stimulating portion may cause agglutination of the living body-derived cells by coating the inner surface of the receiving container with a non-adhesive material to which the living body-derived cells do not adhere and thereby enhancing agglutination of the living body-derived cells without causing adhesion of the cells to the inner surface. Alternatively, the cell-stimulating portion may cause agglutination of the living body-derived cells by grooves formed on the bottom of the receiving container. By doing so, the living body-derived cells can be brought into contact with one another by easily and efficiently agglutinating the living body-derived cells.

In the first aspect, the cell separation apparatus may be configured so that the decomposition treatment unit includes an arm supporting a centrifuge container receiving the cell suspension so as to be swingable around a swing axis and a rotating portion rotating the arm around a predetermined axis away from the swing axis; and the efficiency-increasing mechanism is disposed at a position adjacent to the centrifuge container when the arm is at rest. According to this configuration, the efficiency-increasing portion increases the efficiency of the living body-derived cells, and a concentrated solution is recovered as a final product, which can maximize the therapeutic effect of the final product.

In the above-described configuration, the cell separation apparatus may include a cell-counting portion measuring the number of living body-derived cells contained in the cell suspension and a controller operating the efficiency-increasing portion based on the data of the number of the living body-derived cells measured by the cell-counting portion. By doing so, the cell suspension is treated by the efficiency-increasing portion under more suitable conditions according to the number of the living body-derived cells, and the efficiency of the living body-derived cells can be more effectively increased.

In the first aspect, the cell separation apparatus may be configured so that the efficiency-increasing mechanism includes a cell-counting portion disposed in the conveyance path system conveying the cell suspension from the decomposition treatment unit to the cell concentrating unit and measuring the number of the living body-derived cells contained in the cell suspension; a cell-treating portion disposed in the conveyance path system and increasing the ratio of the living body-derived cells, effective for therapy, contained in the cell suspension; and a controller adjusting the conditions for treating the living body-derived cells with the cell-treating portion based on the number of the living body-derived cells measured by the cell-counting portion.

According to this configuration, the number of living body-derived cells contained in cell suspension is counted by the cell-counting portion during conveyance in the conveyance path system, and the living body-derived cells are treated by the cell-treating portion under conditions appropriately adjusted by the controller according to the measurement results. With this, since the cell suspension in which the ratio of the effective living body-derived cells is increased is conveyed to the cell concentrating unit, the therapeutic effect of the final product can be improved while guaranteeing the sterility.

In the above-described configuration, the cell-treating portion may include a cell-stimulating portion applying stimulation to the living body-derived cells in the cell suspension; and the controller may control the intensity of the stimulation from the cell-stimulating portion. By doing so, the living body-derived cells whose function is decreased can be activated with appropriate stimulation applied to the living body-derived cells. In such a case, the cell-stimulating portion may include an ultrasonic transducer generating ultrasound in the cell suspension, and the controller may adjust the level of the ultrasound output generated by the ultrasonic transducer. Alternatively, the cell-stimulating portion may include a light source irradiating the cell suspension with light, and the controller may adjust the intensity of the light to be emitted from the light source. By doing so, the living body-derived cells can be activated by a simple configuration and operation.

In the above-described configuration, the cell-treating portion may include a cell-sorting channel sorting living body-derived cells from the cell suspension; and the controller may adjust the cell density or the flow rate of the cell suspension flowing in the cell-sorting channel. By doing so, the living body-derived cells among the components contained in the cell suspension are sorted in the sorting channel and are conveyed to the cell concentrating unit, which makes it possible to obtain a concentrate having a higher purity of living body-derived cells.

Here, the cell-sorting channel may have a surface on which an antibody specific to cells other than the effective living body-derived cells is immobilized. By doing so, cells ineffective for therapy are captured by the antibody in the cell-sorting channel merely by allowing the cell suspension to flow in the cell-sorting channel. As a result, the living body-derived cells can be sorted.

Furthermore, the cell-sorting channel may have an inlet through which the cell suspension enters, an outlet disposed on the concentration unit side at a position away from the inlet along the flow direction of the cell suspension and connected to the cell concentrating unit, and an outlet disposed on the waste solution side at a position away from the inlet in the direction oblique to the flow direction of the cell suspension, wherein a plurality of linear adsorption layers on which the antibody is immobilized may be formed on the bottom of the cell-sorting channel so as to obliquely intersect the flow direction of the cell suspension and so as to be approximately parallel to one another with gaps therebetween. By doing so, the cell suspension enters into the cell-sorting channel from the inlet, the living body-derived cells move toward the outlet on the concentrating unit side along the flow direction, and the cells other than the living body-derived cells move toward the outlet on the waste solution side in the direction oblique to the flow direction. With this, the living body-derived cells can be sorted from the cell suspension and can be conveyed to the cell concentrating unit.

The cell-sorting channel may include an ultrasonic transducer generating standing waves of ultrasound in the direction intersecting the flow direction of the cell suspension and include a branching flow path system for distributing the cell suspension at the antinodes of the ultrasound toward the cell concentrating unit and the cell suspension at the nodes of the ultrasound toward a waste solution container. By doing so, among the cells contained in the cell suspension, the living body-derived cells are captured by the ultrasound antinodes and are conveyed to the cell concentrating unit, and the erythrocytes remaining in the cell suspension are captured by the ultrasound nodes and are conveyed to the waste solution container, respectively, through the branching flow path system. By doing so, the living body-derived cells can be sorted from the cell suspension and sent to the cell concentrating unit. Furthermore, since the living body-derived cells are activated by the irradiation with ultrasound, the therapeutic effect of the final product can be further increased.

The second aspect of the present invention relates to a method for activating fat-derived cells including heating fat-derived cells separated from human fat tissue at 38 to 42° C.

According to the second aspect of the present invention, fat-derived cells can be simply activated merely by heating human fat-derived cells at 38 to 42° C. Therefore, fat-derived cells can be activated by a simple operation while guaranteeing the sterility in a noncontact manner. In addition, when the thus-activated fat-derived cells are used in therapy, a high therapeutic effect can be obtained promptly.

The third aspect of the present invention relates to a process of producing a graft material containing fat-derived cells separated from human fat tissue. The process includes the step of activating the fat-derived cells by heating the fat-derived cells at 38 to 42° C.

According to the third aspect of the present invention, since fat-derived cells are simply activated by a simple operation of merely heating fat-derived cells at 38 to 42° C. in the activating step, a graft material that can promptly provide a high therapeutic effect, while guaranteeing sterility, can be produced.

In the third aspect, the process of producing a graft material may further include the step of digesting the fat tissue for separating the fat-derived cells from the fat tissue by stirring the fat tissue together with a digestive enzyme solution, and the step of activating may be performed by stirring the fat tissue together with the digestive enzyme solution at 38 to 42° C. in the step of digesting. By doing so, since the steps of digesting and activating are simultaneously performed, the time necessary for producing the graft material can be shortened.

In the third aspect, the process of producing a graft material may further include the steps of digesting the fat tissue for separating the fat-derived cells from the fat tissue by stirring the fat tissue together with a digestive enzyme solution; and washing the fat-derived cells separated by means of the digestive enzyme solution in the step of digesting with a washing solution, wherein the step of activating may be performed by washing the fat-derived cells with the washing solution having a temperature of 38 to 42° C. in the step of washing. By doing so, the time from the activation of fat-derived cells until the use for therapy is shortened, and the fat-derived cells with higher activity can be used for therapy.

The fourth aspect of the present invention provides a graft material produced by any of the above-described processes of producing a graft material.

What is claimed is:

1. A cell separation apparatus comprising:
a decomposition treatment unit for producing a cell suspension by digesting living body tissue to release living body-derived cells from the living body tissue;
a cell concentrating unit for generating a cell-concentrated solution by concentrating the cell suspension by centrifugation; and
a efficiency-increasing mechanism for increasing the efficiency of the living body-derived cells contained in the cell-concentrated solution.

2. The cell separation apparatus according to claim 1, wherein the efficiency-increasing mechanism comprises:
a receiving container connected to a conveyance path system, which conveys the cell suspension by being connected to the decomposition treatment unit and the cell concentrating unit, and receiving the cell suspension;
a content-measuring portion measuring the content of the receiving container;
a medium-supplying mechanism supplying, to the receiving container, a medium for fractionating erythrocytes and the living body-derived cells contained in the cell suspension;
a medium supply amount calculator calculating the supply amount of the medium to be supplied to the receiving container by the medium-supplying mechanism based on the content measured by the content-measuring portion;
a controller controlling the medium-supplying mechanism to supply the medium based on the supply amount calculated by the medium supply amount calculator; and
a recovery mechanism recovering the living body-derived cells fractionated by the medium in the receiving container while separating the living body-derived cells and the erythrocytes.

3. The cell separation apparatus according to claim 2, wherein the medium is a hemagglutinating agent that agglutinates the erythrocytes.

4. The cell separation apparatus according to claim 3, wherein
the decomposition treatment unit includes a collecting container collecting the living body tissue and a stirring mechanism stirring the inside of the collecting container; and
the receiving container is the collecting container.

5. The cell separation apparatus according to claim 3, wherein the recovery mechanism includes a filter that is disposed between the receiving container of the conveyance path system and the cell concentrating unit and that captures the agglutinated erythrocytes and allows the living body-derived cells to pass through.

6. The cell separation apparatus according to claim 2, wherein the medium is a density-gradient solution having a specific gravity larger than that of the living body-derived cells and smaller than that of the erythrocytes.

7. The cell separation apparatus according to claim 5, wherein
the cell concentrating unit includes a centrifuge container receiving the cell suspension and a centrifuge performing centrifugation using the centrifuge container; and
the receiving container is the centrifuge container.

8. The cell separation apparatus according to claim 2, wherein the content-measuring portion is a scale measuring the weight of the receiving container.

9. The cell separation apparatus according to claim 2, wherein the content-measuring portion is an interface sensor detecting the height position of the interface of the cell suspension in the receiving container.

10. The cell separation apparatus according to claim 2, further comprising an erythrocyte concentration-measuring portion measuring the concentration of erythrocytes contained in the cell suspension,
wherein the medium supply amount calculator calculates a supply amount of the medium based on the erythrocyte concentration measured by the erythrocyte concentration-measuring portion and the content measured by the content-measuring portion.

11. The cell separation apparatus according to claim 2, further comprising a discharge path connected to the bottom of the receiving container and performing discharging from the receiving container,
wherein the recovery mechanism comprises an absorbance sensor measuring absorbance inside the receiving container at different positions in the depth direction and a discharge-amount adjuster adjusting the amount of discharging to the discharge path from the receiving container based on the absorbance measured by the absorbance sensor at each position.

12. The cell separation apparatus according to claim 2, further comprising a washing mechanism washing the living body-derived cells recovered by the recovery mechanism.

13. The cell separation apparatus according to claim 1, wherein the efficiency-increasing mechanism comprises:
a receiving container connected to a conveyance path system, which conveys the cell suspension by being connected to the decomposition treatment unit and the cell concentrating unit, and receiving the cell suspension;
a state-quantity measuring portion measuring a state quantity representing the condition of the cell suspension received in the receiving container;
a cell-stimulating portion stimulating the living body-derived cells contained in the cell suspension received in the receiving container; and
a controller controlling the intensity of the stimulation to be applied to the living body-derived cells by the cell-stimulating portion based on the state quantity measured by the state-quantity measuring portion.

14. The cell separation apparatus according to claim 13, wherein
the state-quantity measuring portion is a temperature sensor measuring the temperature of the cell suspension in the receiving container;
the cell-stimulating portion includes a heater warming the inside of the receiving container; and
the controller controls the temperature of the heater based on the temperature of the cell suspension measured by the temperature sensor.

15. The cell separation apparatus according to claim 14, further comprising a stirring mechanism stirring the inside of the receiving container.

16. The cell separation apparatus according to claim 13, wherein
the state-quantity measuring portion is a liquid level sensor measuring the amount of cell suspension in the receiving container;
the cell-stimulating portion includes an ultrasonic transducer generating ultrasound in the receiving container; and
the controller controls the output level of ultrasound to be generated by the ultrasonic transducer based on the amount of the cell suspension measured by the liquid level sensor.

17. The cell separation apparatus according to claim 13, wherein
the state-quantity measuring portion is an oxygen concentration sensor measuring the oxygen concentration of the cell suspension received in the receiving container;
the cell-stimulating portion includes a low-oxygen-gas supplying portion supplying a gas with an oxygen concentration lower than that of air to the receiving container; and
the controller controls the supply amount of the gas supplied to the receiving container from the low-oxygen-gas supplying portion based on the oxygen concentration measured by the oxygen concentration sensor.

18. The cell separation apparatus according to claim 13, wherein
the state-quantity measuring portion is a liquid level sensor measuring the amount of the cell suspension received in the receiving container;
the cell-stimulating portion includes an activating-agent supplying portion supplying an activating agent that activates the living body-derived cells in the receiving container; and
the controller controls the supply amount of the activating agent to be supplied to the receiving container from the activating-agent supplying portion based on the liquid amount measured by the liquid level sensor.

19. The cell separation apparatus according to claim 18, further comprising an activating agent-removing mechanism for removing the activating agent.

20. The cell separation apparatus according to claim 18, wherein the activating agent is a growth factor or a nitric oxide-inducing factor.

21. The cell separation apparatus according to claim 13, wherein
the state-quantity measuring portion is a cell density meter measuring the density of the living body-derived cells contained in the cell suspension in the receiving container;
the cell-stimulating portion stimulates the living body-derived cells in the cell suspension by bringing the living body-derived cells into contact with one another in the receiving container; and
the controller controls the supply amount of the cell suspension to be supplied to the receiving container through the conveyance path system based on the density measured by the cell density meter.

22. The cell separation apparatus according to claim 21, wherein the cell-stimulating portion causes agglutination of the living body-derived cells by coating the inner surface of the receiving container with a non-adhesive material to which the living body-derived cells do not adhere.

23. The cell separation apparatus according to claim 21, wherein the cell-stimulating portion causes agglutination of the living body-derived cells by grooves formed on the bottom of the receiving container.

24. The cell separation apparatus according to claim 1, wherein
the decomposition treatment unit includes an arm supporting a centrifuge container receiving the cell suspension so as to be swingable around a swing axis and a rotating portion rotating the arm around a predetermined axis away from the swing axis; and
the efficiency-increasing mechanism is disposed at a position adjacent to the centrifuge container when the arm is at rest.

25. The cell separation apparatus according to claim 24, further comprising:
a cell-counting portion measuring the number of living body-derived cells contained in the cell suspension; and
a controller operating the efficiency-increasing portion based on the information of the number of the living body-derived cells measured by the cell-counting portion.

26. The cell separation apparatus according to claim 1, wherein the efficiency-increasing mechanism includes:
a cell-counting portion disposed in the conveyance path system conveying the cell suspension from the decomposition treatment unit to the cell concentrating unit and measuring the number of the living body-derived cells contained in the cell suspension;
a cell-treating portion disposed in the conveyance path system and increasing the ratio of the living body-derived cells, effective for therapy, contained in the cell suspension; and
a controller adjusting the conditions for treating the living body-derived cells with the cell-treating portion based on the number of the living body-derived cells measured by the cell-counting portion.

27. The cell separation apparatus according to claim 26, wherein
the cell-treating portion includes a cell-stimulating portion applying stimulation to the living body-derived cells; and
the controller adjusts the intensity of the stimulation from the cell-stimulating portion.

28. The cell separation apparatus according to claim 27, wherein
the cell-stimulating portion includes an ultrasonic transducer generating ultrasound in the cell suspension; and
the controller adjusts the output level of the ultrasound by the ultrasonic transducer.

29. The cell separation apparatus according to claim 27, wherein
the cell-stimulating portion includes a light source irradiating the cell suspension with light; and
the controller adjusts the intensity of light emitted from the light source.

30. The cell separation apparatus according to claim 26, wherein
the cell-treating portion includes a cell-sorting channel sorting living body-derived cells from the cell suspension; and
the controller adjusts the cell density or the flow rate of the cell suspension flowing in the cell-sorting channel.

31. The cell separation apparatus according to claim 30, wherein the cell-sorting channel has a surface on which an antibody specific to the cells other than the effective living body-derived cells is immobilized.

32. The cell separation apparatus according to claim 31, wherein the cell-sorting channel has an inlet through which the cell suspension enters, an outlet disposed on the concentration unit side at a position away from the inlet along the flow direction of the cell suspension and connected to the cell concentrating unit, and an outlet disposed on the waste solution side at a position away from the inlet in the direction oblique to the flow direction; and a plurality of linear adsorption layers on which the antibody is immobilized is formed on the bottom of the cell-sorting channel so as to obliquely intersect the flow direction of the cell suspension and so as to be approximately parallel to one another with gaps therebetween.

33. The cell separation apparatus according to claim 30, wherein the cell-sorting channel includes:
an ultrasonic transducer generating standing waves of ultrasound in the direction intersecting the flow direction of the cell suspension; and
a branching flow path system for distributing the cell suspension at the antinodes of the ultrasound toward the cell concentrating unit and the cell suspension at the nodes of the ultrasound toward a waste solution container.

34. A method for activating fat-derived cells, comprising heating fat-derived cells separated from human fat tissue at 38 to 42° C.

35. A process of producing a graft material containing fat-derived cells separated from human fat tissue, comprising the step of:
activating the fat-derived cells by heating the fat-derived cells at 38 to 42° C.

36. The process of producing a graft material according to claim 35, further comprising the step of:
digesting the fat tissue for separating the fat-derived cells from the fat tissue by stirring the fat tissue together with a digestive enzyme solution, wherein
the step of activating is performed by stirring the fat tissue together with the digestive enzyme solution at 38 to 42° C. in the step of digesting.

37. The process of producing a graft material according to claim 35, further comprising the steps of:
digesting the fat tissue for separating the fat-derived cells from the fat tissue by stirring the fat tissue together with a digestive enzyme solution; and
washing the fat-derived cells separated by means of the digestive enzyme solution in the step of digesting with a washing solution, wherein
the step of activating is performed by washing the fat-derived cells with the washing solution having a temperature of 38 to 42° C. in the step of washing.

38. A graft material produced by the process of producing a graft material according to claim 35.
